(19) United States
(12) Patent Application Publication
(10) Pub. No.: US 2024/0277353 A1
(43) Pub. Date: Aug. 22, 2024

(54) MEDICAL ASPIRATION SYSTEM

(71) Applicant: Covidien LP, Mansfield, MA (US)

(72) Inventors: Rónán Wood, Galway (IE); Sean Ward, Dublin (IE); Erico Cruz, Galway (IE); Julio Jenaro, Galway (IE); Niall P. Cosgrove, Galway (IE); Alistair Courtney, Belfast (GB); Aishling Hickey, Offaly (IE)

(21) Appl. No.: 18/436,966

(22) Filed: Feb. 8, 2024

Related U.S. Application Data
(60) Provisional application No. 63/485,615, filed on Feb. 17, 2023.

(51) Int. Cl. A61B 17/22; A61B 17/00

(52) U.S. Cl. CPC A61B 17/22; A61B 2017/00022; A61B 2017/00132; A61B 2017/00137

(57) ABSTRACT

An example method includes determining, by control circuitry, a flow rate of a fluid within a catheter lumen fluidically coupled to a valve, and comparing, by the control circuitry, the flow rate to a first flow rate threshold and a second flow rate threshold less than the first flow rate threshold. In response to determining that the flow rate is greater than or equal to the first flow rate threshold, the example method includes controlling, by the control circuitry, the valve to be in a first operational state, and in response to determining that the flow rate is less than or equal to the second flow rate threshold, controlling, by the control circuitry, the valve to be in a second operational state.

This application claims the benefit of U.S. Provisional Patent Application No. 63/485,615, filed on Feb. 17, 2023, and entitled, “MEDICAL ASPIRATION SYSTEM,” the entire content of which is incorporated herein by reference.

### TECHNICAL FIELD

This disclosure relates to medical aspiration.

### BACKGROUND

In some cases, medical aspiration can be used to remove material from a patient. For example, medical aspiration can be used to remove a thrombus, such as a clot or other occlusion, from a blood vessel of a patient.

### SUMMARY

This disclosure describes example devices and systems configured to reduce withdrawal of body fluid (e.g., blood) from a patient during a medical aspiration procedure, and related methods. In examples described herein, an aspiration system is configured to reduce blood loss during a procedure, even with a relatively large aspiration catheter (e.g., 12 French or greater) or any size catheter, by controlling the fluid flow (e.g., controlling the fluid flow rate) and/or controlling the amount of fluid that flows through the aspiration catheter. For example, the aspiration system may monitor fluid flow through the catheter using a sensor and actuate a control valve based on the fluid flow. In some examples, the control valve is configured to allow a relatively higher flow to pass through the catheter lumen when the distal end of the aspiration catheter encounters a blockage (e.g., from thrombus or potentially a vessel wall) and also configured to regulate (e.g., reduce) the flow rate of fluid if the aspiration catheter is in patent blood flow in a vessel. In some examples, the aspiration system is configured to enable a clinician to “search” for thrombus in the vessel using fluoroscopy guidance, while only applying a high or greater suction force when the catheter tip is occluded. Thus, the aspiration system can be configured to provide the suction force that would ordinarily result in the aspiration of relatively high blood volumes in response to determining the catheter tip is occluded.

In some examples described herein, an aspiration system is configured to monitor blood flow within the aspiration system using a sensor and actuate a flow control valve based on the monitored flow. The system will allow a relatively “full” or greater suction force (e.g., unrestricted fluid flow in the catheter lumen) when the distal end of the aspiration catheter encounters a blockage (from thrombus or potentially a vessel wall) but will regulate the application of the suction force if the aspiration catheter is in patent (substantially unblocked) blood flow in a vessel, thereby reducing the aspiration of blood when the catheter is unblocked.

In some examples, the system is configured to actuate the flow control valve based on the determined (e.g., measured or received from another device) flow rate and flow rate thresholds to control an operational state of the system, e.g., valve fully open, valve fully closed, valve partially open, cycling between fully open and closed, cycling between any or all of fully open, fully closed, and/or partially open or closed, cycling between fully open or closed and partially closed and open according to cycling parameters and/or a predetermined cycling schedule, or the like. In some examples, the system determines a current mode of operation based on the determined flow rate and first and second flow rate thresholds, and then controls the valve to be in one of the several operational states.

For example, the system may determine that the aspiration system is in patent blood flow (e.g., unblocked by a thrombus) based on the determined flow rate and the first and second flow rate thresholds, and control the valve to be in a “search mode” comprising periodically opening the valve for a first amount time (state 1) and closing the valve for a second amount of time (state 2) that is greater than the first amount of time. Additionally, the system may determine that the aspiration system is “blocked,” e.g., by a thrombus, based on the determined flow rate and the first and second flow rate thresholds, and control the valve to be in an “aspiration mode” comprising controlling the valve to remain open to apply a high such force to the aspiration catheter while the determined flow rate is less the first flow rate threshold. Additionally, the system may determine that the aspiration system is partially blocked, e.g., by a thrombus, based on the determined flow rate and the first and second flow rate thresholds, and control the valve to be in an “pulse mode” comprising periodically opening the valve for a third amount time (state 3) and closing the valve for a fourth amount of time (state 4) to clear the partial blockage while reducing blood loss in order to do so.

In other examples, the system actuates the flow control valve based on the determined flow rate and a flow rate reference value. For example, the system may dynamically and/or in real-time determine when to open and close the valve, partially or fully, in order to control the valve to control the flow rate of fluid within the aspiration system to be the flow rate reference value, e.g., to reduce and/or minimize the difference between the flow rate of the fluid and the flow rate reference value. In some examples, the system may control the valve to open or close, fully or partially, according to a proportional-integral-derivative (PID) control loop, an adaptive algorithm such as fuzzy logic, a machine learning algorithm and/or method, or the like.

The devices, systems, and techniques of this disclosure may provide one or more advantages and benefits. For instance, allowing for different levels of suction force to be applied for different amounts of time to the catheter lumen may reduce a volume of body fluid withdrawn from the body during an aspiration procedure and provide an automatic and intuitive procedure for reducing blockages in the system. In some examples, the devices, systems, and techniques may provide automatic determination of the size of the aspiration catheter and adjust control of the valve based on catheter size.

In one example, this disclosure describes a method including: determining, by control circuitry, a flow rate of a fluid within a catheter lumen fluidically coupled to a valve; comparing, by the control circuitry, the flow rate to a first flow rate threshold and a second flow rate threshold less than the first flow rate threshold; in response to determining that the flow rate is greater than or equal to the first flow rate threshold, controlling, by the control circuitry, the valve to be in a first operational state; and in response to determining that the flow rate is less than or equal to the second flow rate threshold, controlling, by the control circuitry, the valve to be in a second operational state.

In another example, this disclosure describes a medical aspiration system including: a valve configured to open or close to control a suction force applied to a catheter lumen; and control circuitry configured to: determine a flow rate of a fluid within the catheter lumen; compare the flow rate to a first flow rate threshold and a second flow rate threshold less than the first flow rate threshold; in response to determining that the flow rate is greater than or equal to the first flow rate threshold, control the valve to be in a first operational state; and in response to determining that the flow rate is less than or equal to the second flow rate threshold, control the valve to be in a second operational state.

In another example, this disclosure describes a medical device for aspirating material from a patient, the device including: a suction source; an aspiration catheter defining a lumen fluidically coupled to the suction source; a valve configured to open or close to control a suction force applied to the catheter lumen; and control circuitry configured to control the valve to open or close based on a flow rate, a first flow rate threshold, and a second flow rate threshold.

In another example, this disclosure describes a method including: determining, by control circuitry, a flow rate of a fluid within a catheter lumen fluidically coupled to a valve; comparing, by the control circuitry, the flow rate to a flow rate reference value; determining, by the control circuitry, a duty cycle of a first operational state and a second operational state of the valve based on a difference between the flow rate and the flow rate reference value; and controlling, by the control circuitry, the valve to be in the first and second operational states to control a suction force applied to the catheter lumen according to the duty cycle.

In another example, this disclosure describes a medical aspiration system including: a valve configured to open or close to control a suction force applied to a catheter lumen; and control circuitry configured to: determine a flow rate of a fluid within the catheter lumen fluidically coupled to a valve; compare the flow rate to a flow rate reference value; determine a duty cycle of a first operational state of the valve and a second operational state of the valve based on a difference between the flow rate and the flow rate reference value; and control the valve to be in the first and second operational states to control a suction force applied to the catheter lumen according to the duty cycle.

In another example, this disclosure describes a medical device for aspirating material from a patient, the device including: a suction source; an aspiration catheter defining a lumen fluidically coupled to the suction source; a valve configured to open or close to control a suction force applied to the catheter lumen; and control circuitry configured to control the valve to open or close based on a flow rate and a flow rate reference value.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic diagram illustrating an example aspiration system including a flow control valve.

FIG. 2 is a flow diagram of an example method controlling the flow rate of a fluid in an aspiration system.

FIG. 3 is a plot of example flow rates of different sized catheters and a corresponding example operational state plot of a valve over a period of time.

FIG. 4 is a flow diagram of another example method controlling the flow rate of a fluid in an aspiration system.

FIG. 5 is a flow diagram of another example method controlling the flow rate of a fluid in an aspiration system.

FIG. 6 is a flow diagram of an example method of determining flow rate threshold values for controlling the flow rate of a fluid in an aspiration system.

FIG. 7 is a flow diagram of another example method controlling the flow rate of a fluid in an aspiration system.

FIG. 8 is a plot of an example flow rate over a period of time and a corresponding example operational state plot of a dynamic duty cycle of a valve over the period of time.

FIG. 9 is a plot of another example flow rate and a corresponding example operational state plot of a dynamic duty cycle of a valve over the period of time.

### DETAILED DESCRIPTION

This disclosure describes devices and systems configured to regulate a flow rate of a body fluid, such as blood, from a body of a patient during a medical aspiration procedure, as well as medical aspiration systems (e.g., vascular aspiration systems) including such devices and systems, and corresponding methods. An aspiration catheter can be used to remove a thrombus from a hollow anatomical structure (e.g., a blood vessel) of a patient. For example, a distal opening of the catheter may be positioned in the hollow anatomical structure near a thrombus and an aspiration force can be applied to a lumen of the aspiration catheter in order to draw the thrombus through the catheter lumen and out of the hollow anatomical structure. In some instances, such as with deep vein thrombosis (DVT) and pulmonary embolism (PE) procedures, it may be preferable to use larger aspiration catheters (e.g., 12 French or greater) to remove large clot burdens from vessels. As the aspiration catheter lumen diameter increases, the flow through the aspiration catheter increases, shortening the time in which a maximum blood volume that can be extracted from a patient will be reached more quickly than in a case with a smaller aspiration catheter, and potentially force a premature end to the procedure. Additionally, if the distal tip of an aspiration catheter is not fully engaged with a thrombus as a suction force is being applied to a lumen of the catheter, then blood may be unintentionally aspirated from the patient via the catheter. It is desirable to limit blood loss during an aspiration procedure.

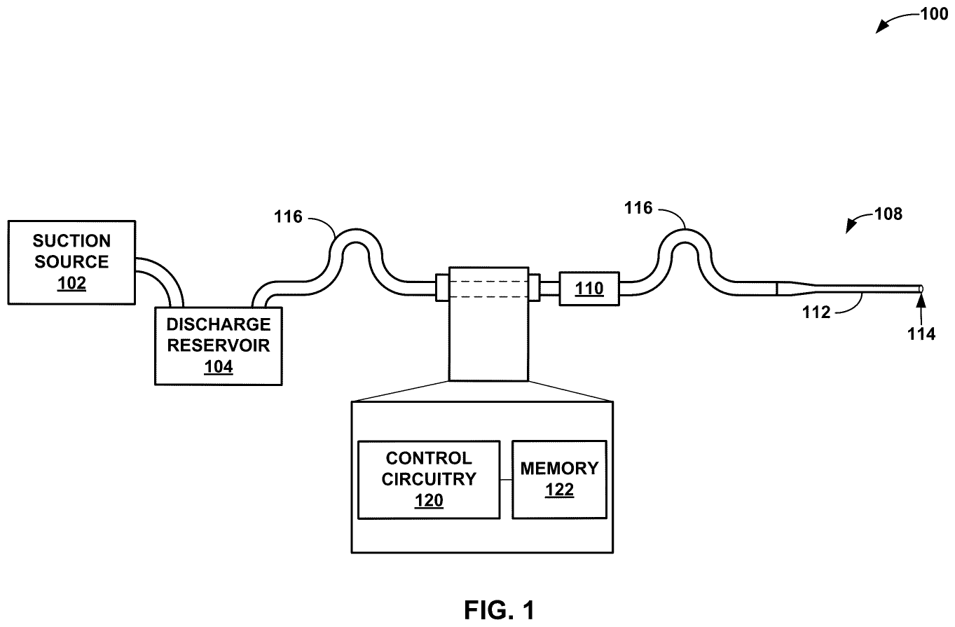

FIG. 1 is a schematic diagram illustrating an example medical aspiration system 100 including a suction source 102, a discharge reservoir 104, a catheter 108 (also referred to herein as “aspiration catheter 108”), and a valve 110. Medical aspiration system 100 may be used to treat a variety of conditions, including thrombosis. Thrombosis occurs when a thrombus (e.g., a blood clot or other material such as plaques or foreign bodies) forms and obstructs vasculature of a patient. For example, medical aspiration system 100 may be used to treat a pulmonary embolism or deep vein thrombosis, which may occur when a thrombus forms in a deep vein of a patient, such as in a leg of the patient.

Medical aspiration system 100 is configured to remove a thrombus from a patient. Medical aspiration system 100 may be configured to remove a thrombus by via catheter 108, e.g., to draw the thrombus from the patient using a suction force applied to catheter 108. Material passing through catheter 108 is deposited into discharge reservoir 104, via a suction force applied by suction source 102 to catheter 108 (e.g., to an inner lumen of catheter 108). Catheter 108 includes an elongated body 112 defining a catheter lumen (not shown in FIG. 1) and terminating in a distal opening 114. To treat a patient with thrombosis, a clinician may position distal opening 114 of catheter 108 in a blood vessel of the patient near the thrombus or other occlusion and apply a suction force (also referred to herein as suction, vacuum force, negative pressure, or aspiration force) to the catheter 108 (e.g., to one or more lumens of the catheter) to engage the thrombus with suction force at distal opening 114 of catheter 108. For example, suction source 102 can be configured to create a negative pressure within the inner lumen of catheter 108 to draw a material from the inside the blood vessel into the catheter lumen via distal opening 114 of catheter 108. The negative pressure within the inner lumen can create a pressure differential between the inner lumen and the environment external to at least a distal portion of catheter 108 that causes the material, e.g., a thrombus, fluid (e.g., blood, saline introduced into the patient as part of the aspiration procedure, or the like), and/or other material, to be introduced from the blood vessel into the catheter lumen via catheter distal opening 114. For example, the fluid may flow from patient vasculature, into the catheter lumen via distal opening 114, and subsequently through aspiration tubing 116 (also referred to herein as “vacuum tube 116”) into discharge reservoir 104.

Once distal opening 114 of aspiration catheter 108 has engaged a thrombus that is within a blood vessel, the clinician may remove aspiration catheter 108 with the thrombus held within opening 114 or attached to the distal tip of elongated body 112, or suction off pieces of the thrombus (or the thrombus as a whole) until the thrombus is removed from the blood vessel of the patient through a lumen of aspiration catheter 108 itself and/or through the lumen of an outer catheter in which aspiration catheter 108 is at least partially positioned. The outer catheter can be, for example, a guide catheter configured to provide additional structural support to the aspiration catheter. In some cases, aspiration of thrombus can be performed concurrently with use of a thrombectomy device, such as a thrombus removal basket, to facilitate removal of thrombus via mechanical thrombectomy as well as via aspiration.

As used herein, “suction force” is intended to include, within its scope, related concepts such as suction pressure, vacuum force, vacuum pressure, negative pressure, and the like. A suction force can be generated by a vacuum, e.g., by creating a partial vacuum within a sealed space (e.g., defined by a housing and/or container) fluidically connected to catheter 108, or by direct displacement of liquid in catheter 108 and/or tubing 116 via (e.g.) a peristaltic pump, or otherwise. Accordingly, suction forces or suction as specified herein can be measured, estimated, computed, etc. without need for direct sensing or measurement of force. A “higher,” “greater,” or “larger” (or “lower,” “lesser,” or “smaller”) suction force described herein may refer to the absolute value of the negative pressure generated by the suction source on a catheter or another component, such as a discharge reservoir 104.

In some examples, suction source 102 can comprise a pump (also referred to herein as “pump 102” or “vacuum source 102”). The suction source 102 can include one or more of a positive displacement pump (e.g., a peristaltic pump, a rotary pump, a reciprocating pump, or a linear pump), a direct-displacement pump (e.g., a peristaltic pump, or a lobe, vane, gear, or piston pump, or other suitable pumps of this type), a direct-acting pump (which acts directly on a liquid to be displaced or a tube containing the liquid), an indirect-acting pump (which acts indirectly on the liquid to be displaced), a centrifugal pump, and the like. An indirect-acting pump can comprise a vacuum pump, which displaces a compressible fluid (e.g., a gas such as air) from the evacuation volume (e.g., discharge reservoir 104, which can comprise a canister), generating suction force on the liquid. Accordingly, the evacuation volume (when present) can be considered part of the suction source. In some examples, suction source 102 includes a motor-driven pump, while in other examples, suction source 102 can include a syringe, and mechanical elements such as linear actuators, stepper motors, etc. As further examples, the suction source 102 could comprise a water aspiration venturi or ejector jet.

Medical aspiration system 100 includes control circuitry 120 configured to control a suction force applied by suction source 102 to catheter 108. For example, control circuitry 120 can be configured to directly control an operation of suction source 102 to vary the suction force applied by suction source 102 to the inner lumen of catheter 108, e.g. by controlling the motor speed, or stroke length, volume or frequency, or other operating parameters, of suction source 102. As another example, control circuitry 120 can be configured to control one or more functions of valve 110. Other techniques for modifying a suction force applied by suction source 102 to the inner lumen of catheter 108 can be used in other examples.

Control circuitry 120, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, control circuitry 120 may include multiple components, such as any combination of one or more microprocessors, one or more DSPs, one or more ASICs, or one or more FPGAs, as well as other discrete or integrated logic circuitry, and/or analog circuitry. In some examples, control circuitry 120 may further include, additionally or alternatively to electric-based processors, one or more controls that operate using fluid motion power (e.g., hydraulic power) in combination with or in addition to electricity. For example, control circuitry 120 can include a fluid circuit comprising a fluid circuit comprising a plurality of fluid passages and switches arranged and configured such that, when a fluid (e.g., liquid or gas) flows through the passages and interacts with the switches, the fluid circuit performs the functionality of control circuitry 120 described herein.

Memory 122 may store program instructions, such as software, which may include one or more program modules, which are executable by control circuitry 120. When executed by control circuitry 120, such program instructions may cause control circuitry 120 to provide the functionality ascribed to control circuitry 120 herein. The program instructions may be embodied in software and/or firmware. Memory 122, as well as other memories described herein, may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Although control circuitry 120 and memory 122 are shown in FIG. 1 as being in a common housing, in other examples, control circuitry 120 and/or memory 122 can be physically separate from each other.

With some aspiration systems, some amount of a body fluid may be incidentally withdrawn during the aspiration procedure. For instance, while approaching and aspirating a thrombus with a distal opening of a catheter, the clinician may incidentally aspirate and remove a volume of the patient's blood, e.g., that is not inherently necessary to withdraw as part of the procedure. Medical aspiration system 100 is configured to reduce the incidental withdrawal of the patient's blood during an aspiration procedure by at least regulating a flow rate of fluid through a catheter lumen of catheter 108, e.g., via control of suction source 102 and/or valve 110 by control circuitry 120.

In examples described herein, control circuitry 120 is configured to determine a flow rate of a fluid within an inner lumen (also referred to as a catheter lumen) of catheter 108 fluidically coupled to valve 110. In some examples, medical aspiration system 100 includes a flow sensor configured to measure the flow rate of a fluid (e.g., blood) within the catheter lumen of catheter 108 (e.g., a lumen defined by elongated body 112), e.g., a flow sensor positioned to measure the flow rate of a fluid within the catheter lumen. For example, a flow sensor may comprise an ultrasound sensor positioned proximate to the lumen of catheter 108 and configured to measure a signal indicative of the flow and/or flow rate of the fluid within the lumen of catheter 108. In some examples, medical aspiration system 100 includes a flow sensor that is configured to measure and/or provide an output representing the flow rate of the fluid within a volume fluidically coupled to the catheter lumen of catheter 108, e.g., within tubing 116 or valve 110, from which the flow rate of the fluid within the lumen of catheter 108 may be determined by control circuitry 120. For example, a flow sensor may comprise a plurality of pressure sensors at a plurality of positions within medical aspiration system 100 (e.g., the lumen of catheter 108, tubing 116, and/or valve 110) and configured to measure a signal (e.g., a pressure difference) indicative of a flow and/or flow rate of a fluid within the lumen of catheter 108. In other examples, medical aspiration system 100 may include sensors, components, and/or parameters that provide output indicative of parameters other than flow rate and based upon which control circuitry 120 may be configured to determine the flow rate of the fluid, e.g., a change in the volume or weight of discharge reservoir 104, control parameters and/or sensors of suction source 102 (e.g., an amount of a negative pressure suction source 102 applied), or any suitable measurements for determining a flow rate within the lumen of catheter 108. Control circuitry 120 is configured to compare the determined flow rate to a first flow rate threshold and a second flow rate threshold less than the first flow rate threshold, and control valve 110 based on the comparisons.

In some examples, control circuitry 120 is configured to control valve 110 to be in a first operational state in response to determining that the flow rate is greater than or equal to the first flow rate threshold. For example, a flow rate greater than or equal to the first flow rate threshold may indicate that medical aspiration system 100 has not captured a thrombus. A measured (e.g., sensed) flow rate of the fluid is greater than or equal to the first flow rate threshold indicates, for example, that fluid is moving within medical aspiration system 100 without being fully blocked, e.g., fluid flow may be unblocked because a thrombus is not captured. The first operational state of valve 110 may be a substantially closed valve state (e.g., fully closed or nearly fully closed) in which valve 110 enables application of a relatively low suction force, which may be a minimum suction force or no suction force (e.g., valve 110 substantially prevents application of a suction force), to the catheter lumen. For example, the first operational state of valve 110 may be a substantially closed valve state in which the valve is fully closed or nearly fully closed, e.g., fully closed to within manufacturing tolerances of the valve. In some example, valve 110, when in the first operational state, may enable a minimum flow rate of 100 milliliters per minute (ml/min) or less, 50 ml/min or less, 10 ml/min or less, 1 ml/min or less, or zero flow. Control circuitry 120 may be configured to control valve 110 to be in the first operational state to substantially reduce or even prevent the fluid from flowing within catheter 108, tubing 116, and valve 110 for a predetermined amount of time, e.g., to reduce an amount of fluid aspirated by medical aspiration system 100 as part of a thrombus search part of a medical procedure.

Control circuitry 120 is further configured to control the valve 110 to be in a second operational state in response to determining that the flow rate is less than or equal to the second flow rate threshold. For example, a flow rate less than or equal to the second flow rate threshold may indicate that medical aspiration system 100 has captured a thrombus, e.g., a thrombus is engaged with distal opening 114 of catheter 108 or is traveling through the catheter lumen of catheter 108 or another lumen in fluid communication with the catheter lumen towards discharge reservoir 104. A measured flow rate of the fluid is less than or equal to the second flow rate threshold can indicate that fluid is slowly moving or not moving within medical aspiration system 100 because fluid flow may be blocked by a captured thrombus, or valve 110 is (or was) closed (e.g., to reduce an amount of fluid aspirated without a thrombus being captured). The second operational state of valve 110 may be a substantially open valve state (e.g., fully open or nearly fully open) in which the valve enables application of a relatively high suction force to the catheter lumen. For example, the second operational state of valve 110 may be a substantially open valve state in which the valve is fully open or nearly fully open, e.g., fully open to within manufacturing tolerances of the valve. For example, valve 110, when in the second operational state, may enable a maximum flow rate of 100 milliliters per minute (ml/min) or more, 500 ml/min or more, 1000 ml/min or more, 3000 ml/min or more, or 5000 ml/min or more. In other examples, the valve 110 may enable any other suitable maximum flow rate. Control circuitry 120 may be configured to control valve 110 to be in the second operational state to cause the application of the relatively high suction force to capture a thrombus and/or enable the fluid to flow within catheter 108, tubing 116, and valve 110 for a predetermined amount of time, e.g., to “search” for a thrombus and/or obtain a subsequent measurement of the flow rate of the fluid within catheter 108, tubing 116, or valve 110.

In other examples, control circuitry 120 is configured to compare the determined flow rate to a flow rate reference value, e.g., rather than flow rate thresholds. For example, rather than searching for a thrombus by opening valve 110 for a predetermined amount of time, measuring the resulting flow, and closing valve 110 for a predetermined amount of “wait” time if a thrombus is not captured (e.g., to reduce the amount of fluid aspirated, but during which medical aspiration system 100 is effectively “blind,” e.g., without a flow rate measurement), medical aspiration system 100 may enable a clinician to search for a thrombus in a more continuous manner with a reduced and/or zero wait time. For example, control circuitry 120 may be configured to aspirate fluid with a continuous target “reference” flow rate. Control circuitry 120 may compare the determined flow rate to the flow rate reference value, and control valve 110 to be in the first operations state or the second operational state to control a suction force applied to the catheter lumen based on the flow rate and a flow rate reference value. In some examples, control circuitry 120 may determine a duty cycle of the first and second operational states based on a difference between the determined flow rate and the flow rate reference value, and control circuitry 120 may control valve 110 to be in the first and second operational states according to the duty cycle.

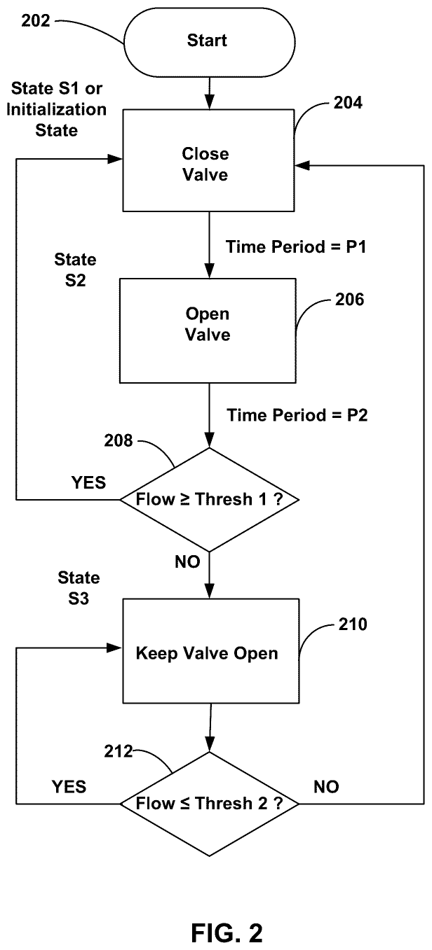

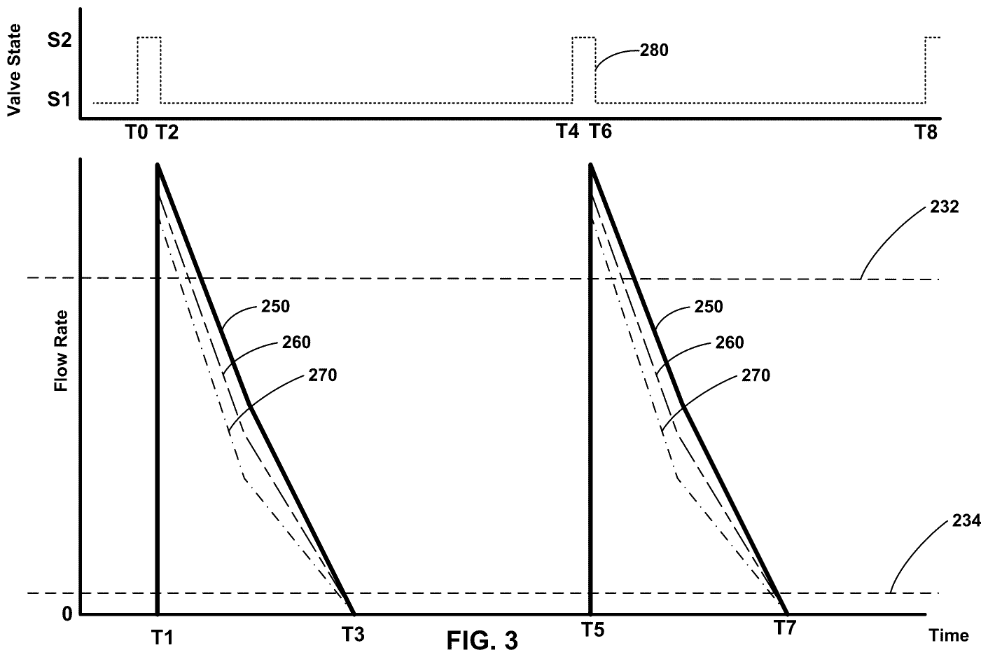

FIG. 2 is a flow diagram of an example method of controlling the flow rate of a fluid in an aspiration system. Although the example technique of FIG. 2 is described with respect to medical aspiration system 100, catheter 108, valve 110, and control circuitry 120, the example technique of FIG. 2 may be performed using any system including an aspiration catheter and valve herein. The example technique of FIG. 2 is also described with respect to plots 250, 260, 270, and 280 of FIG. 3. FIG. 3 is a plot of example flow rates 250, 260, 270 of different sized catheters over a period of time and an example operational state plot 280 illustrating the operational state of valve 110 over the period of time.

A clinician may start an aspiration procedure (202). For example, the clinician may introduce aspiration catheter 108 into vasculature of a patient and distally advance aspiration catheter 108 toward a thrombus within the vasculature of the patient. The clinician may then cause control circuitry 120 to initiate a method of automatically controlling the flow rate of a fluid in medical aspiration system 100 during the procedure, e.g., via a user interface of a device in communication with control circuitry 120.

In accordance with the method of FIG. 2, control circuitry 120 causes and/or otherwise control valve 110 to be in a first operational state S1 (204). For example, and with reference to FIG. 3, valve 110 may be in first operational state S1 before time T0 as illustrated by operational state plot 280 in FIG. 3. In the example shown in FIG. 3, the time scale of operational state plot 280 corresponds to the time scale of flow rate plots 250, 260, and 270. During different portions of the method of FIG. 2 described herein, and as illustrated in FIG. 3, valve 110 may be in a first operational state S1 before time T0, a second operational state S2 between times T0 and T2 and times T4 and T6, and in the first operational state S1 between times T2 and T4 and after time T6 until time T8. Valve 110 may be substantially closed (e.g., fully closed or nearly fully closed to the extent permitted by manufacturing tolerances) in the first operational state S1, e.g., to substantially reduce or even prevent application of a suction force to the catheter 108 lumen (for example, to prevent aspiration of fluid while catheter 108 is being positioned and/or while searching for a thrombus). Valve 110 may be substantially open in the second operational state S2 to enable application of a relatively high suction force to the catheter 108 lumen. In some examples, control circuitry 120 may cause and/or otherwise control valve 110 to be in the first operational state S1 for a first predetermined amount or time, e.g., first time period P1 (FIG. 2). For example, control circuitry 120 may cause a signal to be sent to valve 110, and/or a valve control circuit (not shown), to cause valve 110 to be in the first operational state (e.g., closed).

Control circuitry 120 may then cause and/or control valve 110 to be in the second operational state S2 (206), e.g., at time T0 (FIG. 3). For example, control circuitry 120 may determine that the first predetermined amount of time P1 has passed, and in response may cause and/or control valve 110 to be in the second operations state S2. In some examples, control circuitry 120 may cause and/or control valve 110 to be in the second operational state S2 for a second predetermined amount or time, e.g., second time period P2 (FIG. 2). In the example shown in FIG. 3, the second time period P2 corresponds to the period of time between T0 and T2. In some examples, the first and second time periods P1, P2 may be different from each other, e.g., time period P1 may be greater than time P2. As an example time period P1 can be about 950 milliseconds (ms) and time period P2 can be about 50 ms in some examples. In other examples, the first and second time periods P1, P2 may be the same. In the example shown in FIG. 3, the first time period P1 (e.g., with valve 110 substantially closed) corresponds to the time period between T2 and T4 as described below (e.g., P1 is not fully shown in FIG. 3 for times before time T0, but is shown as being between T2 and T4).

Control circuitry 120 determines a flow rate of a fluid within a catheter lumen of catheter 108. For example, medical aspiration system 100 may include a flow rate sensor (not shown) configured to measure the flow rate of a fluid (e.g., blood) within the lumen of catheter 108 (e.g., the lumen defined by elongated body 112), e.g., a flow rate sensor positioned to measure the flow rate of a fluid within the catheter lumen. For example, a flow sensor may comprise an ultrasound sensor positioned proximate to the lumen of catheter 108 and configured to measure a signal indicative of the flow and/or flow rate of the fluid within the lumen of catheter 108. In some examples, medical aspiration system 100 may include a flow rate sensor configured to measure and/or provide an output representing the flow rate of the fluid within a volume fluidically coupled to the catheter lumen of catheter 108, e.g., within tubing 116 or valve 110, e.g., from which the flow rate of the fluid within the lumen of catheter 108 may be determined by control circuitry 120. For example, a flow sensor may comprise a plurality of pressure sensors at a plurality of positions within medical aspiration system 100 (e.g., the lumen of catheter 108, tubing 116, and/or valve 110) and configured to measure a signal (e.g., a pressure difference) indicative of a flow and/or flow rate of a fluid within the lumen of catheter 108. In other examples, medical aspiration system 100 may include other sensors, components, and/or parameters that provide output indicative of parameters other than flow rate and based upon which control circuitry 120 may be configured to determine the flow rate of the fluid, e.g., a change in the volume or weight of discharge reservoir 104, control parameters and/or sensors of suction source 102 (e.g., an amount of a negative pressure suction source 102 applied), or any suitable measurements for determining a flow rate within the lumen of catheter 108. In some examples, control circuitry 120 determines the flow rate of the fluid within the catheter 108 lumen substantially at or near the end of the second time period, e.g., either at time T2 or just before time T2 so as to make a determination (as described below) whether to cause and/or control valve 110 to be in the first operational state S1 or to remain in the second operational state S2.

Control circuitry 120 compares the determined flow rate of the fluid within the lumen of catheter 108 to a first flow rate threshold 232 (FIG. 3) and a second flow rate threshold 234 (FIG. 3) less than the first flow rate threshold 232 (208). In response to determining that the determined flow rate is greater than or equal to the first flow rate threshold, control circuitry 120 causes and/or otherwise controls valve 110 to be in the first operational state S1 for time period P1 (YES branch of block 208). The method may then proceed at block 204. The method steps corresponding to blocks 204 through the YES branch of block 208 may form a “search cycle” (also referred to as a “search loop” or a “search mode”) of medical aspiration system 100, e.g., while a thrombus is not captured. FIG. 3 illustrates example flow rates 250, 260, 270 of a fluid within medical aspiration system 100 and the corresponding operational state 280 of valve 110 during a first search cycle (T0 to T4) and a second search cycle (T4 to T8).

Alternatively, FIG. 3 illustrates a search cycle from times T2 to T6, which may correspond with the method steps corresponding to blocks 204 through the YES branch of block 208. For example, control circuitry 120 may cause and/or otherwise control valve 110 to be in the first operational state S1 for time period P1, e.g., from T2 to T4, which may be about 950 ms in some examples. P1 may be an amount of time selected to be sufficient to allow a flow within medical aspiration system 100, e.g., within catheter 108, tubing 116, and/or valve 110, to settle and/or stop, and/or a time sufficient for pressures within catheter 108, tubing 116, and/or valve 110 to settle and/or stabilize. In the example shown, just before time T2, flow rates 250, 260, 270 may be relatively high because of suction source 102 may apply a relatively high suction force to the lumen of catheter 108 with valve 110 in the second operational state, e.g., substantially open. When control circuitry 120 causes and/or controls valve 110 to be in the first operations state S1 (substantially closed) at time T2 and method step (204) in response to flow rates 250, 260, 270 being greater than or equal to flow rate threshold 232, the flow rates 250, 260, 270 decrease to substantially zero over a period of time (e.g., not immediately) between times T2 and T3. At time T3, the flow rates 250, 260, 270 may be substantially zero (e.g., zero or nearly zero to the extent permitted by manufacturing tolerances) and substantially stable, and pressure within the lumen of catheter 108 may be substantially stable, e.g., with a substantially zero net motive force on the fluid within the lumen of catheter 108. Control circuitry 120 may then cause and/or control valve 110 to be in the second operational state S2 (substantially open) at time T4 and method step (206) for a second predetermined time period P2, which may be about 50 ms in some examples, allowing suction source 102 to apply a suction force to the lumen of catheter 108.

In some examples, flow rates 250, 260, 270 may not immediately respond, e.g., there may be a delay in initiation of the flow rate of the fluid due to the length of the fluid pathway between suction source 102 and distal opening 114 of catheter 108. In other examples, flow rates 250, 260, 270 may respond very quickly, but there may be a delay in measurement and/or determination of the flow rates by medical aspiration system 100.

As shown in FIG. 3, if medical aspiration system 100 has not captured a thrombus, then flow rates 250, 260, 270 may increase very rapidly at time T5 (after a delay that is less than P2). In the example shown, flow rates 250, 260, 270 rise until valve 110 is closed, e.g., until time T6. In other examples, flow rates 250, 260, 270 may reach a steady state flow (e.g., a constant flow rate) before valve 110 is closed at time T6. Medical aspiration system 100 may then measure a flow rate, and control circuitry 120 may determine, a subsequent flow rate, e.g., a flow rate at or just before time T6, and control circuitry 120 may compare the subsequent flow rate with flow rate threshold 232 (208). If the subsequent flow rate is greater than or equal to flow rate threshold 232, then control circuitry 120 may continue the search cycle again (YES branch of block 208) to continue searching for a thrombus.

The ratio of the second predetermined amount of time to the first predetermined amount of time, e.g., an open/close duty cycle P2/P1, may reduce the amount of fluid aspirated while a thrombus is not captured and while searching for a thrombus. The method of FIG. 2, as well as other methods described herein, may allow for a relatively high flow, higher suction to draw in a thrombus, during a shorter amount of time and with less fluid aspirated than a continuous high suction search or even a lower flow, lower suction continuous search mode. The method may continue through the steps corresponding to blocks 204 to the YES branch of block 208 until a thrombus is captured and/or a user intervenes, e.g., via a user interface of medical aspiration system 100.

Referring back to FIG. 2, in response to determining that the determined flow rate is less than the first flow rate threshold (NO branch of block 208), control circuitry 120 may cause and/or otherwise control valve 110 to be in (or remain in) the second operational state S2 (210). For example, control circuitry 120 may cause a signal to be sent to valve 110, and/or a valve control circuit (not shown), to cause valve 110 to be in the second operational state (e.g., open). Control circuitry 120 may then determine that the flow rate is less than or equal to the second flow rate threshold (YES branch of block 212). For example, a flow rate less than or equal to the second flow rate threshold may indicate that medical aspiration system 100, e.g., catheter 108, has captured a thrombus. In response to determining that the flow rate is less than or equal to the second flow rate threshold, control circuitry 120 causes or otherwise controls valve 110 to be in a second operational state S2 at (210), e.g., to apply a relatively high suction force to the catheter 180 lumen so as to retain and/or aspirate the thrombus. Control circuitry 120 may determine a subsequent flow rate (e.g., via a subsequent measurement) (210), and proceed again to block 212.

Method steps corresponding to blocks 210 through the YES branch of block 212 may form an “aspiration cycle” (also referred to as an “aspiration loop” or an “aspiration mode”) of medical aspiration system 100, e.g., while a thrombus is captured and/or being aspirated. During the aspiration mode, medical aspiration system 100 may determine (e.g., measure) one or more subsequent quantities indicative of the current and/or most recent flow rate within the catheter 108 lumen, tubing 116, and/or valve 110, and control circuitry 120 may determine the one or more subsequent flow rate. Control circuitry 120 may determine the one or more subsequent flow rates at regular intervals, e.g., at a flow measurement sampling rate, at irregular intervals, according to a schedule, or at any suitable time subsequent to the time at which control circuitry 120 determined the prior flow rate. For example, control circuitry 120 may determine the one or more subsequent flow rates at a relatively high flow measurement sampling rate, e.g., with a sampling time that is less than the first or second predetermined amounts of time P1 and P2, e.g., less than 50 ms, 20 ms or less, 10 ms or less, 1 ms or less, or at any suitable flow measurement sampling rate, e.g., so as to reduce an amount of fluid removed from the patient after a thrombus is aspirated and the flow rate within medical aspiration system 100 increases. In some examples, the aspiration mode may be a third operational state S3 of valve 110 in which valve 110 remains substantially open until control circuitry 120 causes and/or controls valve 110 to be in another state, e.g., rather than for a predetermined amount of time.

Control circuitry 120 may determine that the flow rate, or a subsequent flow rate, is greater than the second flow rate threshold (NO branch of block 212). For example, medical aspiration system 100 may aspirate the thrombus, and the blockage may reduce or be removed allowing fluid to flow. In response to determining that the flow rate, or a subsequent flow rate, is greater than the second flow rate threshold, control circuitry 120 may cause and/or otherwise controls valve 110 to be in the first operational state S1 (closed), e.g., for the first predetermined amount of time P1 (204). The method may continue in the search cycle of method steps (204) through the YES branch of (208), the aspiration cycle of method steps (210) through the YES branch of (212), or alternating between the search cycle and aspiration cycle according to the NO branches of (208) and (212) described above, until the method is terminated, e.g., by a clinician via the user interface, or via any other suitable termination event.

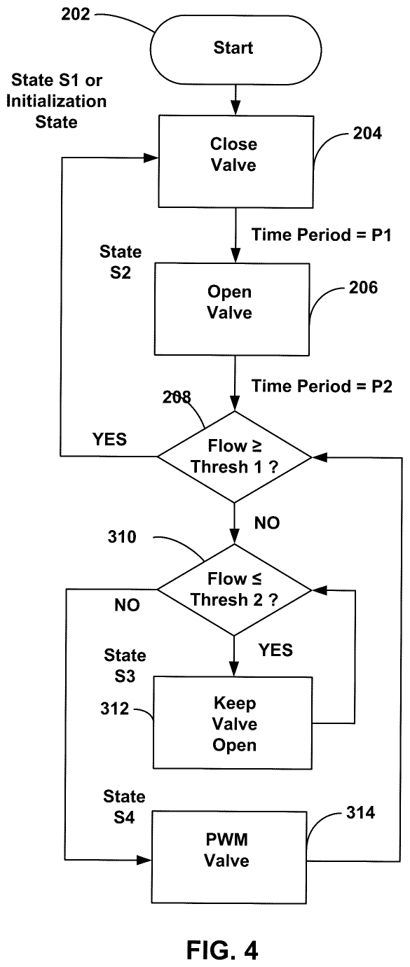

FIG. 4 is a flow diagram of another example method controlling the flow rate of a fluid in an aspiration system. The method of FIG. 4 may be substantially similar to the method of FIG. 2 described above, except that the method of FIG. 4 includes an additional mode and/or operational state S4. Although the example technique of FIG. 4 is described with respect to medical aspiration system 100, catheter 108, valve 110, and control circuitry 120, the example technique of FIG. 4 may be performed using any system including an aspiration catheter and valve herein.

As with the method of FIG. 2, the method of FIG. 4 includes both a search mode and an aspiration mode. For example, a clinician may start an aspiration procedure (202), control circuitry 120 may cause and/or otherwise control valve 110 to be in a first operational state S1 (204) for a first predetermined amount of time P1, control circuitry 120 may cause and/or otherwise control valve 110 to be in a second operational state S2 (206) for a second predetermined amount of time P2, control circuitry 120 may determine a flow rate of a fluid within a catheter lumen of catheter 108 at or substantially near the end of the second predetermined amount of time, and in response to determining that the flow rate is greater than or equal to a first flow rate threshold (YES branch of block 208), and control circuitry 120 may cause and/or otherwise control valve 110 to be the first operational state S1 and the method proceeds at block 204.

In response to determining that the flow rate is less than the first flow rate threshold (NO branch of block 208), control circuitry 120 further determines whether the flow rate is less than or equal to a second flow rate threshold (310), e.g., where the second flow rate threshold is less than the first flow rate threshold. In response to determining that the flow rate is less than or equal to the second flow rate threshold (YES branch of block 310), control circuitry 120 causes and/or otherwise controls valve 110 to be in, or to remain in, the second operational state S2 (open) (312). Control circuitry 120 may determine one or more subsequent flow rates during (312), e.g., at a flow measurement sample rate as described above. Method step (310) may be substantially similar to method step (212) described above, method step (312) may be substantially similar to method step (210) described above, and method steps (312) and the YES branch of (310) may be substantially similar to the aspiration mode, or third operational state S3, as described above.

In response to determining that the flow rate, or a subsequent flow rate, is greater than the second flow rate threshold (NO branch of block 310), control circuitry 120 may controls medical aspiration system 100 (e.g., valve 110) to be in a fourth operational state S4, e.g., a rapid pulse mode or a pulse wave modulation (PWM) mode (314). For example, control circuitry 120 may determine that the flow rate determined in the search mode is less than the first flow rate threshold at (208) but greater than the second flow rate threshold at (310), or a subsequent flow rate determined during the aspiration mode may increase to be greater than the second flow rate threshold during (312). In other words, control circuitry 120 may determine that the currently determined flow rate is between the first and second flow rate thresholds, which may indicate a partial blockage of the catheter 108 lumen, tubing 116, and/or valve 110. Control circuitry 120 may than cause medical aspiration system 100 to be in the fourth operational state S4.

For example, in response to determining that the flow rate, or a subsequent flow rate, is less than the first flow rate threshold and greater than the second flow rate threshold, control circuitry 120 may cause and/or otherwise control valve 110 to alternatingly be in the first operational state S1 (closed) for a third amount of time P3 and to be in the second operational state S2 (open) for a fourth amount of time P4 while the flow rate, or the subsequent flow rate, of the fluid within the lumen of catheter 108 is less than the first flow rate threshold and greater than the second flow rate threshold during the third or fourth amounts of time (314). In some examples, the third and fourth amounts of time are substantially equal to each other, and in other examples the third and fourth amounts of time are different from each other. In some examples, the third and fourth amounts of time may be an open/close duty cycle of valve 110 that is greater than the P2/P1 duty cycle of the search mode. Additionally, the third and fourth amounts of time may be a cycle period that is less than the P1+P2 search cycle period. For example, the third amount of time P3 may be about 300 ms, and the fourth amount of time P4 may be about 300 ms, e.g., a 600 ms cycle period, as opposed to a 1 second (1000 ms) cycle period of a 950 ms P1 and a 50 ms P2 of the search cycle.

Additionally, in response to determining that the flow rate, or a subsequent flow rate, is less than the first flow rate threshold and greater than the second flow rate threshold, control circuitry 120 may determine one or more subsequent flow rates of the fluid within the catheter 108 lumen fluidically coupled to valve 110 at a flow measurement sampling rate (314). The flow measurement sampling rate may be the same as, or different from, the flow measurement sampling rate at (312) during the aspiration mode. In some examples, the flow measurement sampling rate of (314) comprises time intervals between subsequent flow rate samples (e.g., flow rate measurements, flow rate determinations) that are less than the first predetermined amount of time P1, the second predetermined amount of time P2, the third amount of time P3, and the fourth amount of time P4. For example, the flow measurement sampling rate of (314) may be less than 50 ms, 20 ms or less, 10 ms or less, 1 ms or less, or at any suitable flow measurement sampling rate, e.g., so as to clear a partial blockage within medical aspiration system 100 while limiting and/or reducing an amount of fluid aspirated.

In the example of FIG. 4 shown, the NO branches of blocks 208, 310, and 314 may form the PWM mode or fourth operational state S4. In some examples, at (314), control circuitry 120 may cause and/or control valve 110 to be in the first and second operations states for a single P3, P4 cycle, determine one or more subsequent flow rates, and proceed to (208). In other examples, at (314), control circuitry 120 may cause and/or control valve 110 to be in the first and second operations states for more than one P3, P4 cycle, determine one or more subsequent flow rates, and proceed to (208).

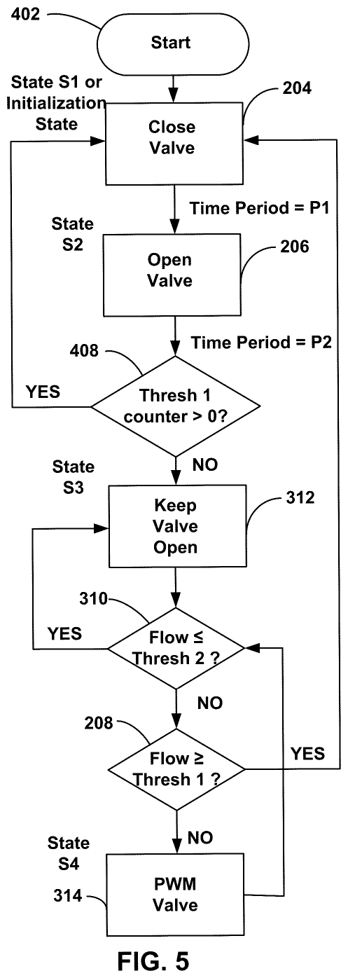

FIG. 5 is a flow diagram of another example method controlling the flow rate of a fluid in an aspiration system. The method of FIG. 5 may be substantially similar to the method of FIG. 4 described above, except that control circuitry 120 may determine the flow rate of the fluid within catheter 108 lumen (or tubing 116 or valve 110) in a continuous manner, e.g., determine one or more subsequent flow rates at a flow measurement sampling rate that is less than the first predetermined amount of time P1 and/or the second predetermined amount of time P2, and the method of FIG. 5 includes a counter decision step (408). Although the example technique of FIG. 5 is described with respect to medical aspiration system 100, catheter 108, valve 110, and control circuitry 120, the example technique of FIG. 5 may be performed using any system including an aspiration catheter and valve herein.

The method of FIG. 5 may provide an increased sensitivity of sensing flow rates greater than or equal to the first flow rate threshold compared to the example methods of FIGS. 2 and 4. For example, there may be latency in any or all of suction source 102 applying a relatively high suction force to the lumen of catheter 108, the response of valve 110, measurement of a quantity indicative of the flow rate by a flow rate sensor or other sensor, or there may be noise in the measurements. In order to reduce the amount of fluid aspirated during a procedure, medical aspiration system 100 may be configured to determine the flow rate within the lumen of catheter 108 substantially continuously during the search cycle, e.g., both of P1 and P2, and only enter the aspiration mode if the flow rate is not greater than or equal to the first flow rate threshold at any time during the last search cycle.

As described above, the method of FIG. 5 may include both the search mode and the aspiration mode. For example, a clinician may start an aspiration procedure (202), control circuitry 120 may cause and/or control valve 110 to be in a first operational state S1 (204) for a first predetermined amount of time P1, and control circuitry 120 may cause and/or control valve 110 to be in a second operational state S2 (206) for a second predetermined amount of time P2.

In the example of FIG. 5 shown, control circuitry 120 determines a flow rate and one or more subsequent flow rates during the first or second predetermined amount of time, e.g., at a flow measurement sampling rate. For example, control circuitry 120 may determine a plurality of flow rates at a flow measurement sampling rate of less than 1000 ms, 500 ms or less, 100 ms or less, 50 ms or less, 20 ms or less, 10 ms or less, 1 ms or less, or at any suitable flow measurement sampling rate during P1 and P2. Control circuitry 120 may determine, e.g., at the flow measurement sampling rate, that the flow rate (e.g., of any of the plurality of flow rate measurements) during the first and second predetermined amounts of time P1, P2 is greater than or equal to the first flow rate threshold, and in response may determine a count of the number of flow rate measurements are greater than or equal to the first flow rate threshold. For example, at the start of P1 at (204), control circuitry 120 may clear a counter variable to zero (e.g., in memory 122), and increment the counter by “1” (or another predetermined value) for each flow rate measurement that control circuitry 120 determines to be greater than or equal to the first flow rate measurement during P1 and/or P2.

In response to determining a flow rate greater than or equal to the first flow rate threshold during the first or second predetermined time periods (e.g., any one flow measurement during P1 or P2), control circuitry 120 may cause and/or otherwise control valve 110 to be in the first operational state S1 (YES branch of block 408). For example, if the counter variable is greater than zero, then control circuitry 120 will cause and/or control valve 110 to be in the first operational state S1 and the method will continue at (204) of FIG. 5.

Control circuitry 120 may determine, e.g., at the flow measurement sampling rate, that a flow rate is not greater than or equal to the first threshold, e.g., that no flow rate during P1 or P2 is greater than or equal to the first flow rate. For example, control circuitry 120 may determine that the flow rate during the first and second predetermined times P1, P2 is less than the first flow rate threshold, and that the counter variable at the end of the search cycle, e.g., P1, P2, is zero. In response to determining that the flow rate is less than the first flow rate threshold during the first or second predetermined time periods (e.g., any one flow measurement during P1 or P2), control circuitry 120 may cause and/or otherwise control valve 110 to be in, or to remain in, the second operational state S1 at (312) (NO branch of block 408).

Control circuitry 120 may then cause and/or otherwise control valve 110 to be in the aspiration mode (third operational state S3) (including the YES branches of blocks 310 and 312) as described above, e.g., after determining the flow rate, or subsequent flow rate, (e.g., the most current flow rate) is less than or equal to the second threshold and indicative of a thrombus being captured by catheter 108. Alternatively, control circuitry 120 may cause and/or otherwise control valve 110 to be in the pulse wave modulation mode (fourth operational state S4) (including the NO branches of blocks 310 and 208) as described above, e.g., after determining the flow rate, or subsequent flow rate, (e.g., the most current flow rate) is less than the first flow rate threshold and the greater than the second flow rate threshold, e.g., between the first and second thresholds and indicative of a partial blockage of the lumen of catheter 108, tubing 116, and/or valve 110.

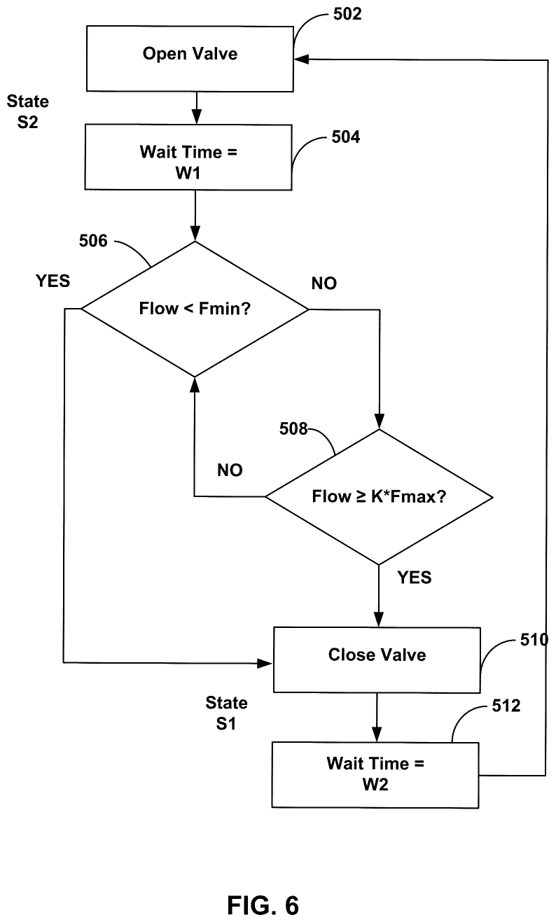

FIG. 6 is a flow diagram of an example method of determining flow rate threshold values for controlling the flow rate of a fluid in medical aspiration system 100. Although the example technique of FIG. 6 is described with respect to medical aspiration system 100, catheter 108, valve 110, and control circuitry 120, the example technique of FIG. 6 may be performed using any system including an aspiration catheter and valve herein. In some examples, the example technique of FIG. 6 may be performed after any one of the example techniques of FIGS. 2, 4, and 5, e.g., after control circuitry 120 has caused and/or controlled valve 110 to be in a search mode for a plurality of cycles.

Medical aspiration system 100 may be configured to automatically determine values for the first and second flow rate thresholds and a size of catheter 108 based on cycling valve 110, e.g., via the technique of FIG. 6. For example, catheter 108 of medical aspiration system may be changed or exchanged, e.g., for a different size catheter 108 having a differing size lumen, e.g., 12 French, 14 French, 16 French, or greater. The technique of FIG. 6 may be used to update the first and second flow rate thresholds based on determined flow rates. The technique of FIG. 6 may be used during an aspiration procedure, e.g., while catheter 108 is within a patient, or the technique of FIG. 6 may be used at any other time, e.g., prior to an aspiration procedure and using a test fluid such as a volume of saline. For example, distal opening 114 of catheter 108 may be submerged in a test fluid for a plurality of valve 110 cycles. In the example shown, a valve cycle comprises at least method steps (502) and (512), e.g., at least completing first and second wait times W1 and W2 and determination of a current flow rate during first wait time W1, as described below. In some examples, a valve cycle may comprise at least one opening and closing of valve 110.

Control circuitry 120 may cause and/or otherwise control valve 110 to be in the second operational state S2 (open) (502) for a first wait time W1 (504). For example, control circuitry 120 may cause and/or otherwise control valve 110 to be substantially open to enable application of a relatively high suction force to the catheter 108 lumen for 50 ms, although other wait times W1 can be used in other examples. In some examples, first wait time W1 may be long enough for a flow to substantially stabilize within medical aspiration system 100, e.g., with the catheter 108 lumen, tubing 116, and/or valve 110.

Control circuitry 120 determines whether a current flow rate is less than or equal to a minimum flow rate Fmin (506). For example, control circuitry 120 may determine the current flow rate, e.g., based on a quantity measured by a sensor such as a flow rate sensor or other sensor from which a flow rate may be determined as described above, and control circuitry 120 may compare the current flow rate with a predetermined minimum flow rate Fmin. The current flow rate may be the most recent flow rate determined by control circuitry 120, of a plurality of flow rates determined by control circuitry 120 during the technique of FIG. 6. For example, a sensor may measure a quantity indicative of a flow rate at a plurality of times, e.g., at a flow measurement sampling rate, and control circuitry 120 may determine a flow rate at each of the plurality of times, e.g., at the flow measurement sampling rate, the most recent of which may be the “current flow rate.” In some examples, the flow measurement sampling rate may comprise a sampling period between measurements/determinations of the flow rate that is less than one or both of the first and second wait times W1 and W2. For example, control circuitry 120 may determine the flow rate at a flow measurement sampling rate of 10 ms, and wait times W1 and W2 may be 50 ms and 950 ms, respectively.

Fmin may be a flow rate indicative of a blockage of the lumen of catheter 108. In some examples, the value of Fmin may be independent of the size of catheter 108. In some examples, the second flow rate threshold may be based on Fmin. If the current flow rate is less than or equal to Fmin (YES branch of bock 506), control circuitry 120 may cause and/or control valve 110 to be in the first operational state S1 (closed) (510) for a second wait time W2 (512). Control circuitry 120 may cause and/or control valve 110 to close for second wait time W2 (e.g., 950 ms although other second wait times W2 can be used in other examples).

If the current flow rate is greater than Fmin (NO branch of block 506), control circuitry 120 may determine whether the current flow rate is greater than or equal to a scaled maximum flow rate K*Fmax (508), where the scaled maximum flow rate K*Fmax comprises a maximum flow rate, Fmax, multiplied by scaling factor K, where K is between 0 and 1. Scaling factor K may represent a flow rate, relative to the maximum flow rate Fmax, indicative of a partial blockage. For example, for K=0.8, flow rates greater than or equal to 80% of the maximum flow rate Fmax may indicate substantially no blockage of the lumen of catheter 108, and a flow rate at which valve 110 should be closed to reduce the amount of fluid aspirated during a procedure. Conversely, flow rates less than 80% of the maximum flow rate Fmax may indicate a partial blockage (e.g., if the flow rate is also above the second flow rate threshold) or a substantially complete blockage of the lumen of catheter 108 (e.g., if the flow rate is less than or equal to the second flow rate threshold), and valve 110 should be opened, or remain open, to aspirate the blockage.

In some examples, adjusting the value of scaling factor K may adjust the amount of excess fluid aspirated during a procedure and/or the efficiency of aspirating a blockage or partial blockage (e.g., a thrombus). For example, smaller values of K may reduce the amount of blood aspirated, but may not efficiently aspirate partial blockages, and larger values of K may ensure that all blockages (full and partial) may be aspirated, but while aspirating a relatively larger amount of fluid during a procedure.

The maximum flow rate Fmax may be determined based on one or more flow rates determined by control circuitry 120, e.g., during one or more cycles of the method of FIG. 6. In some examples, Fmax may be a maximum flow rate of a plurality of previous cycles, or a subset of the plurality of previous cycles. For example, during one or more cycles, control circuitry 120 may determine the maximum flow rate Fmax based on the most recent M determined flow rates of a total N plurality of determined flow rates, where M is an integer value that is less than an integer value N.

For example, control circuitry 120 may determine a plurality of flow rates at a flow measurement sampling rate having a 10 ms period between samples, starting during first wait time W1, e.g., 45 ms after the start of wait time W1. At the first cycle, control circuitry 120 may have determined only the one flow rate, e.g., the current flow rate, and may set the current flow rate to be the maximum flow rate Fmax. At a subsequent cycle, or if control circuitry 120 determines flow rates starting at 35 ms after the start of wait time W1, control circuitry 120 may have determined a plurality of flow rates, and may determine the maximum flow rate to be the largest determined flow rate value of the N most recently determined flow rates, e.g., the last 5 flow rates, or the last 10 flow rates, or the last 100 flow rates.

In some examples of the method of FIG. 6, control circuitry 120 determines the first flow rate threshold to be equal to the scaled maximum flow rate K*Fmax. If the catheter 108 is changed for a larger sized catheter, then control circuitry 120 may determine larger flow rates and determine a larger maximum flow rate Fmax based on the N most recently determined flow rates. If the catheter 108 is changed for a smaller sized catheter, then control circuitry 120 may determine smaller flow rates and determine a smaller maximum flow rate Fmax based on the N most recently determined flow rates. Scaling factor K may reduce the first flow rate threshold value from the maximum flow rate Fmax, e.g., there may be noise in the actual flow rate of an unobstructed lumen of catheter 108 causing variation in actual flow rate, or in the measurement of the actual flow rates. Scaling factor K, as well as thresholds and other values described herein can be, for example, stored in memory 122 of system 100 or a memory of another device.

If the current flow rate is greater than or equal to the first flow rate threshold, e.g., the scaled maximum flow rate K*Fmax (YES branch of block 508), then control circuitry 120 causes and/or otherwise controls valve 110 to be in the first operational state S1 (closed) (510) for a second wait time W2 (512). Control circuitry 120 may cause and/or control valve 110 to close for second wait time W2 (e.g., 950 ms although other second wait times W2 can be used in other examples).

The method may then proceed to (502) for a subsequent cycle. For example, if catheter 108 is changed to a larger sized catheter, then the current flow rate may be larger than Fmax, and control circuitry 120 may adjust Fmax to increase, but it may not yet be the true Fmax, e.g., not all of the N most recently determined flow rates correspond to the new catheter 108. The technique of FIG. 6 may cycle back to (502), after having closed valve 110 for wait time W2 (e.g., to stabilize flow rates and/or pressures within medical aspiration system 100) and control circuitry 120 may determine a one or more subsequent flow rates during (504) and/or (506), and further adjust Fmax at (508) based on the subsequent flow rates. The technique may continue to cycle from (502) to (512) until control circuitry 120 determines that Fmax has stabilized, or for a predetermined number of cycles, e.g., ensuring that the N most recently determined flow rates have stabilized.

If the current flow rate is less than the scaled maximum flow rate K*Fmax (the NO branch at 508), then the method may proceed to (506) where control circuitry 120 may determine whether the current flow rate is less than or equal to the minimum flow rate Fmin. For example, control circuitry 120 may have happened to have determined a new flow rate (e.g., now the “current” flow rate) between (506) and (508), and a blockage may have been encountered. Alternatively, catheter 108 may have been changed to a smaller sized catheter, and the current flow rate, although from an unobstructed lumen and causing control circuitry 120 to determine Fmax to be smaller, may still be less than K*Fmax, and the method may loop between (506) and (508) for a plurality of measurements at the flow measurement sampling rate for a plurality of flow rate determinations until control circuitry 120 adjusts Fmax (based on the N most recently determined flow rates) such that the current flow rate is greater than or equal to K*Fmax, and control circuitry 120 may set the new K*Fmax as the new first flow rate threshold.

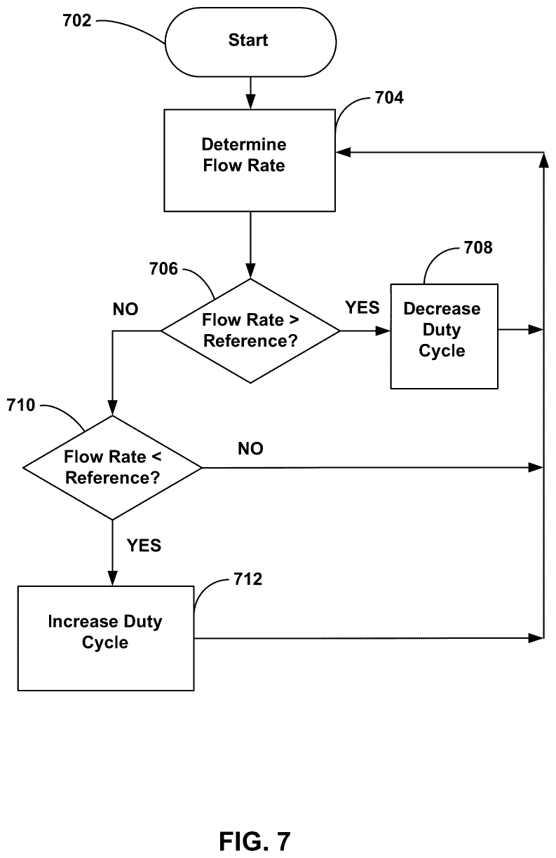

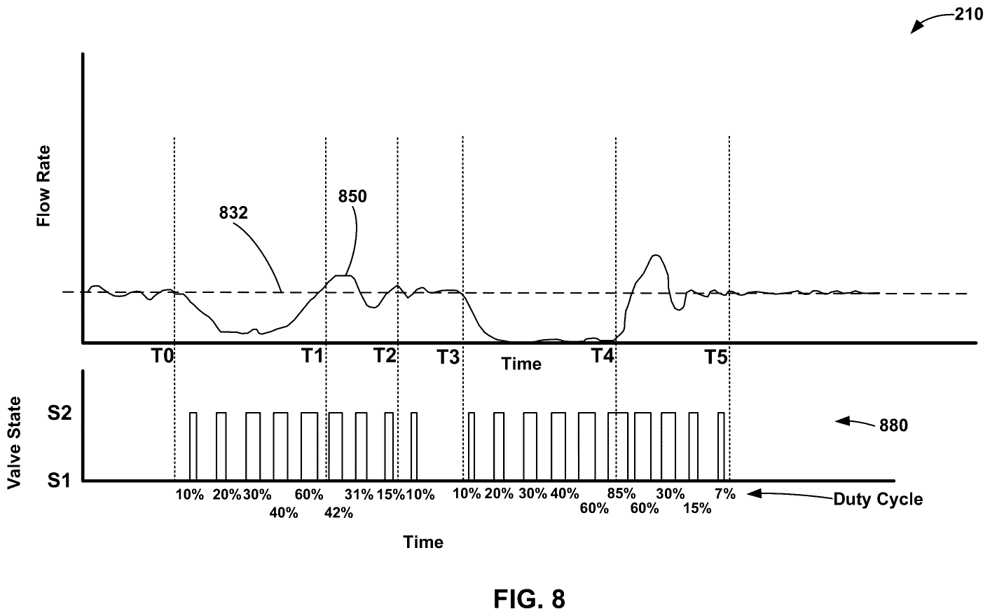

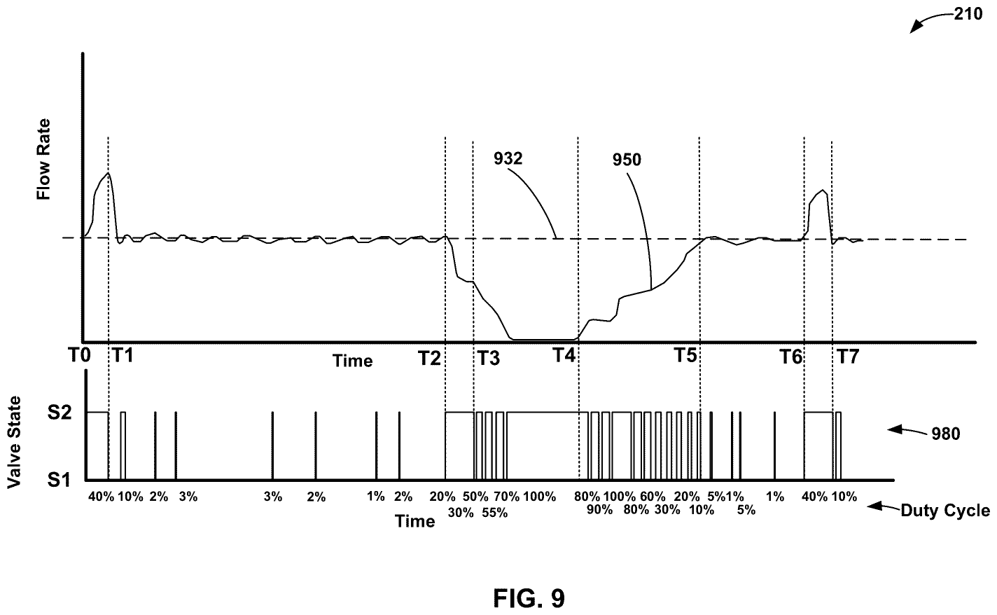

FIG. 7 is a flow diagram of an example method controlling the flow rate of a fluid in an aspiration system. Although the example technique of FIG. 7 is described with respect to medical aspiration system 100, catheter 108, valve 110, and control circuitry 120, the example technique of FIG. 7 may be performed using any system including an aspiration catheter and valve herein. The example technique of FIG. 7 is also described with respect to plots 850 and 880 of FIG. 8 and plots 950 and 980 of FIG. 9. FIG. 8 is a plot of an example flow rate 850 over a period of time and a corresponding example operational state plot 880 of a dynamic duty cycle of valve 110 over the period of time. FIG. 9 is a plot of an example flow rate 950 over a period of time and a corresponding example operational state plot 980 of a dynamic duty cycle of valve 110 over the period of time.

In some examples, FIG. 7 illustrates an example of an adaptive algorithm for controlling a flow rate of a fluid within medical aspiration system 100, e.g., to reduce the amount of fluid aspirated when a thrombus is not captured and/or being aspirated. In some examples, the technique of FIG. 7 may use a proportional-integral-derivative (PID) method and associated PID controller to control the fluid flow rate based on a reference fluid flow rate. In other examples, the technique of FIG. 7 may use machine learning, e.g., fuzzy logic, to control the fluid flow rate based on a reference fluid flow rate.

In some examples, control circuitry 120 (e.g., which may comprise and/or be in communication with a PID controller) is configured to determine a duty cycle of valve 110 based on an amount of a difference between a flow rate within the lumen of catheter 108 in a continuous manner, e.g., determining a dynamic duty cycle via determining the flow at a flow measurement sampling rate, and to control a duty cycle of valve 110 in a substantially continuous manner based on the determined duty cycle. In some examples, the period of a cycle of valve 110 may be 1 second or less, 100 ms or less, 10 ms or less, 1 ms or less, 0.1 ms or less, or any suitable cycling period. The duty cycle comprises a ratio of the period of time that the valve is in the second operational state (open) versus in the first operational state (closed). For example, for a 10 ms cycling period and a 70% duty cycle, control circuitry 120 controls valve 110 to be in the second operational state for 7 ms and in the first operational state for 3 ms.

As described herein, increasing the duty cycle comprising increasing the amount of time of the cycling period that control circuitry 120 controls valve 110 to be in the second operational state and decreasing the amount of time valve 110 is in the first operational state. For example, increasing the duty cycle increases the amount of time in which valve 110 enables application of a relatively high suction force to the catheter 108 lumen, and decreasing the duty cycle decreases the amount of time in which valve 110 enables application of a relatively low suction force to the catheter 108 lumen (or substantially prevents the application of a suction force to the catheter 108 lumen). In some examples, increasing the duty cycle comprises increasing the amount of time of the cycling period that control circuitry 120 controls valve 110 to be in the second operational state, e.g., up to 100% of the time of the cycling period, and decreasing the duty cycle comprises decreasing the amount of time of the cycling period that control circuitry 120 controls valve 110 to be in the first operational state, e.g., down to 0% of the time of the cycling period. In some examples, control circuitry 120 may be configured to control valve 110 in a PWM mode with a cycling period of about 1 microsecond or less, a cycling period of about 10 microseconds or less, a cycling period of about 100 microseconds or less, a cycling period of about 500 microseconds or less, a cycling period of about 1 millisecond or less, a cycling period of about 10 milliseconds or less, a cycling period of about 10 milliseconds or less, a cycling period of about 100 milliseconds or less, a cycling period of about 500 milliseconds or less, a cycling period of about 1 second or less, or any suitable cycling period amount of time.

In some examples, the flow rate reference value is greater than a minimum flow rate detectable by a sensor. For example, a flow rate sensor, or a sensor configured to measure a parameter from which a flow rate may be determined (e.g., a weight and/or weight difference of discharge reservoir 104), may have a flow rate noise amount, e.g., an amount of noise in a direct measurement of the flow rate or an amount of noise in measuring a parameter from which the flow rate may be determined. The flow rate noise amount may limit the resolution, dynamic range, and minimum detectable flow rate of the flow rate sensor. FIG. 8 illustrates a flow rate reference value 832 that is greater than the flow rate noise amount. When the flow rate reference value is greater than the flow rate noise amount of the flow rate sensor, control circuitry 120 may control valve 110 with relative short duty cycle periods, e.g., 1 ms or less, or 0.1 ms or less.

In some examples, it may be desirable to further reduce the amount of fluid aspirated during a procedure, and the flow rate reference value may be less than or equal to the flow rate noise amount of the flow rate sensor. FIG. 9 illustrates a flow rate reference value 932 that is less than or equal to the flow rate noise amount. When the flow rate reference value is less than or equal to the flow rate noise amount of the flow rate sensor, control circuitry 120 may control valve 110 to allow a flow (e.g., via enabling application of a relatively high suction force to the lumen of catheter 108) momentarily, e.g., to periodically increase the flow rate to a level greater than the flow rate noise amount and determined the flow rate, e.g., with a greater measurement sensitivity and/or accuracy, and is some examples to check if there is a blockage and/or captured thrombus. For example, control circuitry 120 may periodically (e.g., every 1000 ms) cause and/or control valve 110 to be in the second operational state (open) for a relatively short pulse, e.g., 50 ms although other pulse widths can be used in other examples.

Referring to FIG. 7, a clinician starts an aspiration procedure (702). For example, and as described above at FIG. 2, the clinician may introduce aspiration catheter 108 into vasculature of a patient and distally advance aspiration catheter 108 toward a thrombus within the vasculature of the patient. The clinician may then cause control circuitry 120 to initiate a method of automatically controlling the flow rate of a fluid in medical aspiration system 100 during the procedure, e.g., via a user interface of a device in communication with control circuitry 120.

Control circuitry 120 determines a flow rate of a fluid within a catheter lumen of catheter 108 (704). For example, medical aspiration system 100 may include a flow rate sensor (not shown) configured to measure the flow rate of a fluid (e.g., blood) within the lumen of catheter 108 (e.g., the lumen defined by elongated body 112), e.g., a flow rate sensor positioned to measure the flow rate of a fluid within the lumen. For example, a flow sensor may comprise an ultrasound sensor positioned proximate to the lumen of catheter 108 and configured to measure a signal indicative of the flow and/or flow rate of the fluid within the lumen of catheter 108. In some examples, medical aspiration system 100 may include a flow rate sensor that is configured to measure and/or provide an output representing the flow rate of the fluid within a volume fluidically coupled to the catheter lumen of catheter 108, e.g., within tubing 116 or valve 110, from which the flow rate of the fluid within the lumen of catheter 108 may be determined by control circuitry 120. For example, a flow sensor may comprise a plurality of pressure sensors at a plurality of positions within medical aspiration system 100 (e.g., the lumen of catheter 108, tubing 116, and/or valve 110) and configured to measure a signal (e.g., a pressure difference) indicative of a flow and/or flow rate of a fluid within the lumen of catheter 108. In other examples, medical aspiration system 100 may include other sensors, components, and/or parameters other than flow rate and based upon which control circuitry 120 may determine the flow rate of the fluid, e.g., a change in the volume or weight of discharge reservoir 104, control parameters and/or sensors of suction source 102 (e.g., an amount of a negative pressure suction source 102 applied), or any suitable measurements for determining a flow rate within the lumen of catheter 108. In some examples, control circuitry 120 determines the flow rate of the fluid within the catheter 108 lumen substantially continuously, e.g., at a relatively high frequency flow measurement sampling rate. In some examples, control circuitry 120 may determine the flow rate at a flow measurement sampling rate having a period between samples (e.g., determined flow rates) of 100 ms or less, 10 ms or less, 1 ms or less, 0.1 ms or less, or any suitable flow measurement sampling rate.

Control circuitry 120 may compare the determined flow rate of the fluid within the lumen of catheter 108 to a flow rate reference value 832, 932 (FIGS. 8, 9) (706). In response to determining that the determined flow rate is greater than flow rate reference value 832, 932 (YES branch of block 706), control circuitry 120 determines a difference between the determined flow rate and flow rate reference value 832, 932. Control circuitry 120 may then determine a duty cycle of the first and second operational states S1, S2 based on the difference between the flow rate and the flow rate reference value. In some examples, control circuitry 120 may additionally determine a cycling period of valve 110. In some examples, if control circuitry 120 has previously determined a duty cycle, then control circuitry 120 modifies the duty cycle to increase the amount of time the valve is in the first operational state and decrease the amount of time the valve is in the second operational state at (708).

In some examples, control circuitry 120 modifies the duty cycle by an amount that is proportional to the difference between the determined flow rate and flow rate reference value 832, 932. For example, in response to determining that the flow rate is greater than the flow rate reference value by a first difference amount, control circuitry 120 may modify the duty cycle by a first duty cycle change amount, and in response to determining that the flow rate is greater than the flow rate reference value by a second difference amount that is greater than the first difference amount, control circuitry 120 may modify the duty cycle by a second duty cycle amount that is greater than the first amount. In some examples, decreasing the duty cycle by the first duty cycle amount comprises increasing an amount of time the valve is in the first operational state by a first amount of time and decreasing an amount of time the valve is in the second operational state by the first amount of time, and decreasing the duty cycle by the second duty cycle amount comprises increasing the amount of time the valve is in the first operational state by a second amount of time that is greater than the first amount of time and decreasing an amount of time the valve is in the second operational state by the second amount of time.

Control circuitry 120 may then control valve 110 to be in the first and second operational states S1, S2 to control a suction force applied to the catheter lumen according to the duty cycle. For example, control circuitry 120 may decrease the duty cycle (708) based on the flow rate being greater than flow rate reference value 832, 932 by at least increasing the amount of time valve 110 is in the first operational state S1 and decreasing the amount of time valve 110 is in the second operations state S2. Control circuitry 120 may then determine a subsequent flow rate of a fluid within a catheter lumen of catheter 108 at (704), e.g., the method loops back to (704).

In response to determining that the determined flow rate is not greater than flow rate reference value 832, 932 (NO branch of block 706), control circuitry 120 may determine that the determined flow rate is less than flow rate reference value 832, 932 (YES branch of block 710)). In response to determining that the determined flow rate is less than flow rate reference value 832, 932 (YES branch of block 710), control circuitry 120 may determine a difference between the determined flow rate and flow rate reference value 832, 932. Control circuitry 120 may then determine a duty cycle of the first and second operational states S1, S2 based on the difference between the flow rate and the flow rate reference value. In some examples, control circuitry 120 may additionally determine a cycling period of valve 110. In some examples, if control circuitry 120 has previously determined a duty cycle, then control circuitry 120 may modify the duty cycle to decrease the amount of time the valve is in the first operational state and increase the amount of time the valve is in the second operational state at (712).

Control circuitry 120 may then cause or otherwise control valve 110 to be in the first and second operational states S1, S2 to control a suction force applied to the catheter lumen according to the duty cycle. For example, control circuitry 120 may increase the duty cycle (712) based on the flow rate being less than flow rate reference value 832, 932 by at least decreasing the amount of time valve 110 is in the first operational state S1 and increasing the amount of time valve 110 is in the second operations state S2. Control circuitry 120 may then determine a subsequent flow rate of a fluid within a catheter lumen of catheter 108 at (704), e.g., the method loops back to (704).

In some examples, as analogously described above for a determined flow rate that is greater than flow rate reference value 832, 932, control circuitry 120 may modify the duty cycle by an amount that is proportional to the difference between the determined flow rate and flow rate reference value 832, 932. For example, in response to determining that the flow rate is less than the flow rate reference value by a first difference amount, control circuitry 120 may modify the duty cycle by a first duty cycle change amount, and in response to determining that the flow rate is greater than the flow rate reference value by a second difference amount that is greater than the first difference amount, control circuitry 120 may modify the duty cycle by a second duty cycle change amount that is greater than the first duty cycle change amount. In some examples, increasing the duty cycle by the first duty cycle change amount comprises decreasing an amount of time the valve is in the first operational state by a first amount of time and increasing an amount of time the valve is in the second operational state by the first amount of time, and increasing the duty cycle by the second amount comprises decreasing the amount of time the valve is in the first operational state by a second amount of time that is greater than the first amount of time and increasing an amount of time the valve is in the second operational state by the second amount of time.

In response to determining that the determined flow rate is not less than the flow rate reference value 832, 932, e.g., substantially equal to flow rate reference value 832, 932 (NO of block 710), control circuitry 120 may leave the duty cycle unchanged and then determine a subsequent flow rate of a fluid within a catheter lumen of catheter 108 at (704), e.g., the method loops back to (704). In some examples, the technique of FIG. 7 may repeat at the flow measurement sampling rate, e.g., (704), (706), and (708) may be a loop that occurs substantially at the flow measurement sampling rate, and (704), (706), (710), and (712) (described below) may be a loop that occurs substantially at the flow measurement sampling rate.

In some examples, control circuitry 120 may cause or otherwise control the open and closed state of valve 110 at the determined duty cycle and a cycling period that is less than the period of the flow measurement sampling rate, e.g., control circuitry 120 may open and close valve 110 faster than the flow measurement sampling rate. In other examples, control circuitry 120 may cause or otherwise valve 110 at the determined duty cycle and a cycling period that is greater than or equal to the period of the flow measurement sampling rate, e.g., control circuitry 120 may open and close valve 110 slower than, or at about the same rate as, the flow measurement sampling rate. In some example, control circuitry 120 may determine a subsequent duty cycle (via looping back to (704) at the flow measurement sampling rate) before completing the current cycle, e.g., if the cycling period is greater than the period of the flow measurement sampling rate. Control circuitry 120 may then immediately control valve 110 according to the subsequent duty cycle rather than completing the previously determined duty cycle.

In some examples, control circuitry 120 may determine a duty cycle substantially continuously and/or a substantially “instantaneous” duty cycle as a function of time, e.g., analogous to a continuous functions as opposed to a discrete function. Control circuitry 120 may control the flow rate of the fluid within the lumen of catheter 108 substantially continuously via controlling the duty cycle of valve 110 substantially continuously.

In some example, control circuitry 120 may cause or otherwise valve 110 to be in the first operational state S1 or the second operational state S2 according to the duty cycle by controlling the valve according to a proportional-integral-derivative (PID) control loop or an adaptive algorithm. The technique of FIG. 7 may include repeating (704), (706), and (708) and/or (704), (706), (710), and (712) in a loop as shown in FIG. 7, as an adaptive or PID control loop. For example, control circuitry 120 may determine and/or modify a duty cycle based on machine learning (e.g., fuzzy) logic in addition to a difference amount between the determined flow rate and flow rate reference value 832, 932. For example, control circuitry 120 may be configured to learn how much to change the duty cycle based on a particular difference amount also based on past determinations, e.g., whether the flow rate is above flow rate reference value 832, 932 and was previously decreasing and/or increasing, and/or based on past results, e.g., how much a particular duty cycle changed a subsequent flow rate in the past.

In some examples, control circuitry 120 may be configured to open and/or close valve 110 a partial amount, e.g., 10% open/90% closed, for an amount of time. For example, control circuitry 120 may determine and/or modify an amount time valve 110 is in a particular partially open state, and the amount valve 110 is partially open, based on the difference between the determined flow rate and the flow rate reference value, and to control valve 110 open and close according to the partial amount.

In some examples, control circuitry 120 may be configured to determine flow rate reference value 832, 932 based on a size of the catheter 108 lumen. For example, control circuitry 120 may determine a larger flow rate reference value 832, 932 for a larger catheter 108 lumen and a smaller flow rate reference value 832, 932 for a smaller catheter 108.

Referring to FIG. 8, control circuitry 120 may determine the flow rate 950 of a fluid within catheter 108 between times T0 and T1 to be less than flow rate reference value 832. For example, the lumen of catheter 108 may be obstructed, or partially obstructed, by a thrombus. Control circuitry 120 may determine a duty cycle according to operational state plot 880 as a function of time and control valve 110 to be in the first and second operational states according to operational state plot 880 between times T0 and T1, e.g., according to (704), (706), (710), and (712) described above, to increase the duty cycle and increase the suction force applied to the lumen to aspirate the blockage/thrombus (and bring the flow rate back to the flow rate reference value 832). At time T1, the blockage may pass through the lumen between times T1 and T2, and the flow rate 850 consequently increases. Control circuitry 120 may decrease and/or increase the duty cycle, e.g., according to (704), (706), (708) and/or (704), (706), (710), (712) to stabilize the flow rate in the lumen at flow rate reference value 832.

At times T2 to T3, there may be no blockage, and control circuitry 120 may determine a relatively stable, and relatively low (e.g., 10%) duty cycle in stabilizing the flow rate of the unobstructed lumen to be substantially close to the relatively low flow rate reference value 832. At time T3, catheter 108 may encounter and capture a thrombus, which may fully block the lumen. Control circuitry 120 may increase the duty cycle per (704), (706), (710), (712), again to aspirate the thrombus, as seen by the increasing duty cycle values between times T3 and T4. At time T4, the thrombus may pass and/or move through the lumen, and the flow rate may rapidly increase overshooting flow rate reference value 832, and control circuitry 120 may again decrease and/or increase the duty cycle, e.g., according to (704), (706), (708) and/or (704), (706), (710), (712) to stabilize the flow rate in the lumen at flow rate reference value 832 by time T5. For example, the duty cycle decreases to a relatively low duty cycle in order to aspirate a relatively low amount of fluid when catheter 108 no longer has a thrombus captured to aspirate, via stabilizing the flow rate within the lumen to be substantially close to the relatively low flow rate reference value 832.

FIG. 9 may be substantially similar to FIG. 8 described above, except that the flow rate reference value 932 may be lower than a flow rate noise amount, and control circuitry 120 may periodically open valve 110 to state 2 for a short amount of time, e.g., 50 ms. In the example shown, control circuitry 120 may cause and/or control valve 110 to be in the second operational state S2 (open), which may increase the duty cycle of operational state 980, e.g., to 40%. In some examples, control circuitry 120 may cause and/or control valve 110 to be in the second operations state S2 (substantially open) for a predetermined amount of time, e.g., from T0 to T1. The flow rate 950 then increases between times T0 and T1, e.g., in order for control circuitry 120 to determine a reliable flow rate and/or determine the flow rate with increased sensitivity and/or accuracy. Between times T1 and T2, the lumen of catheter 108 may be unobstructed and control circuitry 120 may then increase and/or decrease the duty cycle, e.g., according to (704), (706), (708) and/or (704), (706), (710), (712), and based on determining that a thrombus has not been captured between times T0 and T1. In the example shown, control circuitry 120 may control valve 110 with a substantially low duty cycle, e.g., 0%-3% for most of the time between T1 and T2, to stabilize the flow within the lumen of catheter 108 to be the substantially low flow rate reference value 932, even if the uncertainty in the measurements of the flow rate have increased, e.g., because the flow rate is less than the flow rate noise amount of the sensor measurements on which the flow rate is determined.

At time T2, control circuitry 120 causes and/or otherwise controls valve 110 to open to check for a thrombus and/or to increase the sensitivity and/or accuracy measuring the flow rate, and the flow rate 950 decreases rather than increases, indicating the catheter 108 has captured a thrombus. Control circuitry 120 may increase the duty cycle per (704), (706), (710), (712), again to aspirate the thrombus, as seen by the larger duty cycle values between times T3 and T4. At time T4, the thrombus may begin to pass, and control circuitry 120 may then increase and/or decrease the duty cycle, e.g., according to (704), (706), (708) and/or (704), (706), (710), (712). For example, although the sensor noise may increase the uncertainty in the determination of the flow rate 950, control circuitry 120 may still be able to determine that the flow rate is increasing and control the flow rate via controlling the duty cycle of valve 110 accordingly and stabilize the flow rate between times T5 and T6. Between times T6 and T7, control circuitry 120 may cause and/or control valve 110 to be in the second operational state S2 (open) similar to times T0 to T1, e.g., to check for a thrombus and/or to increase flow rate 950 to measure the flow rate with an increased sensitivity and/or accuracy.

The techniques described in this disclosure, including those attributed to control circuitry 120, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the control circuitry 120, instead of or in addition to the partially or wholly digital hardware and/or software described herein. Accordingly, analog or digital hardware may be employed, or a combination of the two. Whether implemented in digital or analog form, or in a combination of the two, control circuitry 120 can comprise a timing circuit.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

The following clauses provide some examples of the disclosure. The examples described herein may be combined in any permutation or combination.

Example 1: A method including: determining, by control circuitry, a flow rate of a fluid within a catheter lumen fluidically coupled to a valve; comparing, by the control circuitry, the flow rate to a first flow rate threshold and a second flow rate threshold less than the first flow rate threshold; in response to determining that the flow rate is greater than or equal to the first flow rate threshold, controlling, by the control circuitry, the valve to be in a first operational state; and in response to determining that the flow rate is less than or equal to the second flow rate threshold, controlling, by the control circuitry, the valve to be in a second operational state.

Example 2: The method of example 1, wherein the first operational state comprises a substantially closed valve state in which the valve enables application of a relatively low suction force to the catheter lumen, and wherein the second operational state comprises a substantially open valve state in which the valve enables application of a relatively high suction force to the catheter lumen.

Example 3: The method of example 1 or example 2, further including: determining, by the control circuitry, that a first predetermined amount of time has passed with the valve in the first operational state; and in response to determining, by the control circuitry, that the first predetermined amount of time has passed with the valve in the first operational state, controlling the valve to be in the second operational state for a second predetermined amount of time.

Example 4: The method of example 3, wherein the first predetermined amount of time is about 950 milliseconds (ms), wherein the second predetermined amount of time is about 50 ms, and wherein determining the flow rate comprises measuring the flow rate after second predetermined amount of time.

Example 5: The method of any one of examples 1 through 4, further including: in response to determining that the flow rate is less than the first flow rate threshold: determining, by the control circuitry and at a flow measurement sampling rate, a subsequent flow rate of the fluid within the catheter lumen; determining, by the control circuitry and at the flow measurement sampling rate, whether the subsequent flow rate of the fluid within the catheter lumen is greater than the second flow rate threshold; and in response to determining that the subsequent flow rate is greater than the second flow rate threshold, controlling, by the control circuitry, the valve to be in a first operational state.

Example 6: The method of example 5, wherein the flow measurement sampling rate is about 10 ms.

Example 7: The method of any one of examples 1 through 6, wherein determining the flow rate comprises directly measuring the flow rate of the fluid within the catheter lumen.

Example 8: The method of any one of examples 1 through 7, wherein determining the flow rate comprises indirectly measuring the flow rate of the fluid within the catheter lumen by at least measuring the flow rate of the fluid within a volume fluidically coupled to the catheter lumen.

Example 9: The method of any one of examples 1 through 8, wherein controlling the valve to be in the first operational state comprises controlling the valve to be in the first operational state for a first predetermined amount of time, and wherein controlling the valve to be in the second operational state comprises controlling the valve to be in the second operational state for a second predetermined amount of time, the method further including: in response to determining that the flow rate is less than the first flow rate threshold and greater than the second flow rate threshold: determining, by the control circuitry and at a flow measurement sampling rate, a subsequent flow rate of the fluid within the catheter lumen; and controlling, by the control circuitry, the valve to alternatingly be in the first operational state for a third amount of time and to be in the second operational state for a fourth amount of time while the subsequent flow rate of the fluid within the catheter lumen is less than the first flow rate threshold and greater than the second flow rate threshold during the third or fourth amounts of time.

Example 10: The method of example 9, wherein the third amount of time and the fourth amount of time are substantially equal.

Example 11: The method of example 9 or example 10, wherein the flow measurement sampling rate comprises time intervals that are less than the first predetermined amount of time, the second predetermined amount of time, the third amount of time, and the fourth amount of time.

Example 12: The method of example 11, wherein the flow measurement sampling rate is about 10 ms, wherein the third and fourth amounts of time are about 300 ms.

Example 13: The method of any one of examples 1 through 12, wherein controlling the valve to be in the first operational state comprises controlling the valve to be in the first operational state for a first predetermined amount of time, the method further including: controlling, by the control circuitry, the valve to be in the second operational state for a second predetermined amount of time; determining, by the control circuitry and at a flow measurement sampling rate, that the flow rate during the first or second predetermined amount of time is greater than or equal to the first flow rate threshold; and controlling, by the control circuitry, the valve to be in the first operational state in response to determining the flow rate during the first or second predetermined amount of time is greater than or equal to the first flow rate threshold.

Example 14: The method of any one of examples 1 through 13, wherein controlling the valve to be in the first operational state comprises controlling the valve to be in the first operational state for a first predetermined amount of time, the method further including: controlling, by the control circuitry, the valve to be in the second operational state for a second predetermined amount of time; determining, by the control circuitry and at a flow measurement sampling rate, that the flow rate during the first or second predetermined amount of time is less than the first flow rate threshold; and controlling, by the control circuitry, the valve to alternatingly be in the first operational state for a third amount of time and to be in the second operational state for a fourth amount of time while a subsequent flow rate of the fluid within the catheter lumen is less than the first flow rate threshold and greater than the second flow rate threshold during the third or fourth amounts of time.

Example 15: The method of example 14, wherein the flow measurement sampling rate comprises time intervals that are less than the first predetermined amount of time, the second predetermined amount of time, the third amount of time, and the fourth amount of time.

Example 16: The method of example 14 or example 15, wherein the third amount of time and the fourth amount of time are substantially equal.

Example 17: The method of any one of examples 1 through 16, wherein the flow rate includes a first flow rate, the method further including: controlling, by the control circuitry, the valve to alternatingly be in the first and second operational states for a plurality of valve cycles, wherein each valve cycle of the plurality of valve cycles comprises: the first operational state in which the valve is closed to reduce a suction force applied to the catheter lumen for a first predetermined amount of time; and the second operational state in which the valve is open to increase the suction force applied to the catheter lumen for a second predetermined amount of time; determining, by the control circuitry, a subsequent flow rate during or after of the second predetermined amount of time; and determining, by the control circuitry, at least one of the first flow rate threshold or the second flow rate threshold based on the subsequent flow rate during the plurality of valve cycles.

Example 18: The method of example 17, further including: determining, by the control circuitry during a valve cycle of the plurality valve cycles, that the subsequent flow rate is less than the first flow rate threshold; and decreasing the first flow rate threshold to be less than or equal to the subsequent flow rate in response to determining that the subsequent flow rate is less than the first flow rate threshold.

Example 19: The method of example 17 or example 18, further including: determining, by the control circuitry during a valve cycle of the plurality valve cycles, that the subsequent flow rate is greater than a maximum flow rate; and increasing the first flow rate threshold based on the subsequent flow rate.

Example 20: The method of any one of examples 17 through 19, further including: determining, by the control circuitry, a size of the catheter lumen based on a maximum flow rate during the plurality of valve cycles.

Example 21: A medical aspiration system including: a valve configured to open or close to control a suction force applied to a catheter lumen; and control circuitry configured to: determine a flow rate of a fluid within the catheter lumen; compare the flow rate to a first flow rate threshold and a second flow rate threshold less than the first flow rate threshold; in response to determining that the flow rate is greater than or equal to the first flow rate threshold, control the valve to be in a first operational state; and in response to determining that the flow rate is less than or equal to the second flow rate threshold, control the valve to be in a second operational state.

Example 22: The medical aspiration system of example 21, wherein the first operational state comprises a substantially closed valve state in which the valve enables application of a relatively low suction force to the catheter lumen, and wherein the second operational state comprises a substantially open valve state in which the valve enables application of a relatively high suction force to the catheter lumen.

Example 23: The medical aspiration system of example 21 or example 22, wherein the control circuitry is further configured to: determine that a first predetermined amount of time has passed with the valve in the first operational state; and in response to determining that the first predetermined amount of time has passed with the valve in the first operational state, control the valve to be in the second operational state for a second predetermined amount of time.

Example 24: The medical aspiration system of example 23, wherein the first predetermined amount of time is about 950 milliseconds (ms), wherein the second predetermined amount of time is about 50 ms, and wherein determining the flow rate comprises measuring the flow rate after second predetermined amount of time.

Example 25: The medical aspiration system of any one of examples 21 through 24, wherein the control circuitry is further configured to: in response to determining that the flow rate is less than the first flow rate threshold: determine, at a flow measurement sampling rate, a subsequent flow rate of the fluid within the catheter lumen; determine, at the flow measurement sampling rate, whether the subsequent flow rate of the fluid within the catheter lumen is greater than the second flow rate threshold; and in response to determining that the subsequent flow rate is greater than the second flow rate threshold, control the valve to be in a first operational state.

Example 26: The medical aspiration system of example 25, wherein the flow measurement sampling rate is about 10 ms.

Example 27: The medical aspiration system of any one of examples 21 through 26, wherein determining the flow rate comprises directly measuring the flow rate of the fluid within the catheter lumen.

Example 28: The medical aspiration system of any one of examples 21 through 27, wherein determining the flow rate comprises indirectly measuring the flow rate of the fluid within the catheter lumen by at least measuring the flow rate of the fluid within a volume fluidically coupled to the catheter lumen.

Example 29: The medical aspiration system of any one of examples 21 through 28, wherein controlling the valve to be in the first operational state comprises controlling the valve to be in the first operational state for a first predetermined amount of time, wherein controlling the valve to be in the second operational state comprises controlling the valve to be in the second operational state for a second predetermined amount of time, wherein the control circuitry is further configured to: in response to determining that the flow rate is less than the first flow rate threshold and greater than the second flow rate threshold: determine, at a flow measurement sampling rate, a subsequent flow rate of the fluid within the catheter lumen; and control the valve to alternatingly be in the first operational state for a third amount of time and to be in the second operational state for a fourth amount of time while the subsequent flow rate of the fluid within the catheter lumen is less than or equal to the first flow rate threshold and greater than or equal to the second flow rate threshold during the third or fourth amounts of time.

Example 30: The medical aspiration system of example 29, wherein the third amount of time and the fourth amount of time are substantially equal.

Example 31: The medical aspiration system of any of example 29 or example 30, wherein the flow measurement sampling rate comprises time intervals that are less than the first predetermined amount of time, the second predetermined amount of time, the third amount of time, and the fourth amount of time.

Example 32: The medical aspiration system of example 31, wherein the flow measurement sampling rate is about 10 ms, wherein the third and fourth amounts of time are about 200 ms.

Example 33: The medical aspiration system of any one of examples 21 through 32, wherein controlling the valve to be in the first operational state comprises controlling the valve to be in the first operational state for a first predetermined amount of time, and wherein the control circuitry is further configured to: control the valve to be in the second operational state for a second predetermined amount of time; determine, at a flow measurement sampling rate, that the flow rate during the first or second predetermined amount of time is greater than or equal to the first flow rate threshold; and control the valve to be in the first operational state in response to determining the flow rate during the first or second predetermined amount of time is greater than or equal to the first flow rate threshold.

Example 34: The medical aspiration system of any one of examples 21 through 33, wherein controlling the valve to be in the first operational state comprises controlling the valve to be in the first operational state for a first predetermined amount of time, and wherein the control circuitry is further configured to: control the valve to be in the second operational state for a second predetermined amount of time; determine, at a flow measurement sampling rate, that the flow rate during the first or second predetermined amount of time is less than the first flow rate threshold; and determine, at the flow measurement sampling rate, a subsequent flow rate of the fluid within the catheter lumen; and control the valve to alternatingly be in the first operational state for a third amount of time and to be in the second operational state for a fourth amount of time while the subsequent flow rate of the fluid within the catheter lumen is less than or equal to the first flow rate threshold and greater than or equal to the second flow rate threshold during the third or fourth amounts of time.

Example 35: The medical aspiration system of example 34, wherein the flow measurement sampling rate comprises time intervals that are less than the first predetermined amount of time, the second predetermined amount of time, the third amount of time, and the fourth amount of time.

Example 36: The medical aspiration system of example 34 or example 35, wherein the third amount of time and the fourth amount of time are substantially equal.

Example 37: The medical aspiration system of any one of examples 21 through 36, wherein the flow rate includes a first flow rate, and wherein the control circuitry is further configured to: control the valve to alternatingly be in the first and second operational states for a plurality of valve cycles, wherein each valve cycle of the plurality of valve cycles comprises: the first operational state in which the valve is closed to reduce the suction force applied to the catheter lumen for a first predetermined amount of time; and the second operational state in which the valve is open to increase the suction force applied to the catheter lumen for a second predetermined amount of time; determine a subsequent flow rate during or after of the second predetermined amount of time; and determine at least one of the first flow rate threshold or the second flow rate threshold based on a second flow rate during the plurality of valve cycles.

Example 38: The medical aspiration system of example 37, wherein the control circuitry is further configured to: determine, during a valve cycle of the plurality valve cycles, that the subsequent flow rate is less than the first flow rate threshold; and decrease the first flow rate threshold to be less than or equal to the subsequent flow rate in response to determining that the subsequent flow rate is less than the first flow rate threshold.

Example 39: The medical aspiration system of example 37 or example 38, wherein the control circuitry is further configured to: determine, during a valve cycle of the plurality valve cycles, that the subsequent flow rate is greater than a maximum flow rate; and increasing the first flow rate threshold based on the subsequent flow rate.

Example 40: The medical aspiration system of any one of examples 37 through 39, wherein the control circuitry is further configured to determine a size of the catheter lumen based on a maximum flow rate of the plurality of cycles.

Example 41: The medical aspiration system of any one of examples 37 through 40, further including: a suction source; and an elongated body defining the catheter lumen.

Example 42: A medical device for aspirating material from a patient, the device including: a suction source; an aspiration catheter defining a lumen fluidically coupled to the suction source; a valve configured to open or close to control a suction force applied to the catheter lumen; and control circuitry configured to control the valve to open or close based on a flow rate, a first flow rate threshold, and a second flow rate threshold.

Example 43: A method includes: determining, by control circuitry, a flow rate of a fluid within a catheter lumen fluidically coupled to a valve; comparing, by the control circuitry, the flow rate to a flow rate reference value; determining, by the control circuitry, a duty cycle of a first operational state and a second operational state of the valve based on a difference between the flow rate and the flow rate reference value; and controlling, by the control circuitry, the valve to be in the first and second operational states to control a suction force applied to the catheter lumen according to the duty cycle.

Example 44: The method of example 43, wherein the first operational state comprises a substantially closed valve state in which the valve enables application of a relatively low suction force to the catheter lumen, and wherein the second operational state comprises a substantially open valve state in which the valve enables application of a relatively high suction force to the catheter lumen.

Example 45: The method of example 44, further including: determining, by the control circuitry, that the flow rate is greater than the flow rate reference value; and modifying, by the control circuitry, the duty cycle to increase an amount of time the valve is in the first operational state and decrease an amount of time the valve is in the second operational state in response to determining that the flow rate is greater than the flow rate reference value.

Example 46: The method of example 45, wherein modifying the duty cycle comprises decreasing, in response to determining that the flow rate is greater than the flow rate reference value by a first difference amount, the duty cycle by a first duty cycle change amount, wherein modifying the duty cycle comprises decreasing, in response to determining that the flow rate is greater than the flow rate reference value by a second difference amount that is greater than the first difference amount, the duty cycle by a second duty cycle change amount that is greater than the first duty cycle change amount, wherein decreasing the duty cycle by the first amount comprises increasing the amount of time the valve is in the first operational state by a first amount of time and decreasing an amount of time the valve is in the second operational state by the first amount of time, and wherein decreasing the duty cycle by the second amount comprises increasing an amount of time the valve is in the first operational state by a second amount of time that is greater than the first amount of time and decreasing an amount of time the valve is in the second operational state by the second amount of time.

Example 47: The method of any one of examples 44 through 46, further including: determining, by the control circuitry, that the flow rate is less than the flow rate reference value; and modifying, by the control circuitry, the duty cycle to decrease an amount of time the valve is in the first operational state and increase an amount of time the valve is in the second operational state in response to determining that the flow rate is less than the flow rate reference value.

Example 48: The method of example 47, wherein modifying the duty cycle comprises increasing, in response to determining that the flow rate is less than the flow rate reference value by a first difference amount, the duty cycle by a first duty cycle change amount, wherein modifying the duty cycle comprises increasing, in response to determining that the flow rate is less than the flow rate reference value by a second difference amount that is greater than the first difference amount, the duty cycle by a second duty cycle change amount that is greater than the first duty cycle change amount, wherein increasing the duty cycle by the first duty cycle change amount comprises decreasing an amount of time the valve is in the first operational state by a first amount of time and increasing an amount of time the valve is in the second operational state by the first amount of time, and wherein increasing the duty cycle by the second duty cycle change amount comprises decreasing an amount of time the valve is in the first operational state by a second amount of time that is greater than the first amount of time and increasing an amount of time the valve is in the second operational state by the second amount of time.

Example 49: The method of any one of examples 42 through 48, wherein determining the flow rate comprises determining the flow rate based on a flow rate measurement from a flow rate sensor, wherein the flow rate reference value is greater than a flow rate noise amount of the flow rate sensor.

Example 50: The method of any one of examples 42 through 49, wherein determining the flow rate comprises determining the flow rate based on a flow rate measurement from a flow rate sensor, and wherein the flow rate reference value is less than or equal to a flow rate noise amount of the flow rate sensor, and wherein the flow rate is a first flow rate, the method further including: controlling, by the control circuitry, the valve to be in the second operational state for a predetermined amount of time; determining, by the control circuitry using the flow rate sensor and within the predetermined amount of time, a second flow rate of the fluid within the catheter lumen; and controlling, by the control circuitry, the valve to be in the first operational state or to be in the second operational state to control the suction force applied to the catheter lumen based on the second flow rate and the flow rate reference value.

Example 51: The method of example 50, wherein the duty cycle is a first duty cycle, the method further including: determining, by the control circuitry, a second duty cycle of the first and second operational states based on a difference between the second flow rate and the flow rate reference value; and controlling, by the control circuitry, the valve to be in the first operational state or to be in the second operational state according to the second duty cycle.

Example 52: The method of any one of examples 42 through 51, wherein controlling the valve to be in the first operational state or the second operational state according to the duty cycle comprises controlling the valve according to at least one of proportional-integral-derivative (PID) control loop or an adaptive algorithm.

Example 53: The method of any one of examples 42 through 52, further including: determining, by the control circuitry, an amount to partially open or close the valve based on the difference between the flow rate and the flow rate reference value; and controlling, by the control circuitry, the valve to open and close according to the partial amount.

Example 54 The method of any one of examples 42 through 53, further including:

Example 55: A medical aspiration system including: a valve configured to open or close to control a suction force applied to a catheter lumen; and control circuitry configured to: determine a flow rate of a fluid within the catheter lumen fluidically coupled to a valve; compare the flow rate to a flow rate reference value; determine a duty cycle of a first operational state of the valve and a second operational state of the valve based on a difference between the flow rate and the flow rate reference value; and control the valve to be in the first and second operational states to control a suction force applied to the catheter lumen according to the duty cycle.

Example 56: The medical aspiration system of example 55, wherein the first operational state comprises a substantially closed valve state in which the valve enables application of a relatively low suction force to the catheter lumen, and wherein the second operational state comprises a substantially open valve state in which the valve enables application of a relatively high suction force to the catheter lumen.

Example 57: The medical aspiration system of example 56, wherein the control circuitry is further configured to: determine that the flow rate is greater than the flow rate reference value; and modify the duty cycle to increase an amount of time the valve is in the first operational state and decrease an amount of time the valve is in the second operational state in response to determining that the flow rate is greater than the flow rate reference value.

Example 58: The medical aspiration system of example 57, wherein modifying the duty cycle comprises decreasing, in response to determining that the flow rate is greater than the flow rate reference value by a first difference amount, the duty cycle by a first duty cycle change amount, wherein modifying the duty cycle comprises decreasing, in response to determining that the flow rate is greater than the flow rate reference value by a second difference amount that is greater than the first difference amount, the duty cycle by a second duty cycle change amount that is greater than the first duty cycle change amount, wherein decreasing the duty cycle by the first amount comprises increasing the amount of time the valve is in the first operational state by a first amount of time and decreasing the amount of time the valve is in the second operational state by the first amount of time, and wherein decreasing the duty cycle by the second amount comprises increasing an amount of time the valve is in the first operational state by a second amount of time that is greater than the first amount of time and decreasing an amount of time the valve is in the second operational state by the second amount of time.

Example 59: The medical aspiration system of any one of examples 56 through 58, wherein the control circuitry is further configured to: determine that the flow rate is less than the flow rate reference value; and modify the duty cycle to decrease an amount of time the valve is in the first operational state and increase an amount of time the valve is in the second operational state in response to determining that the flow rate is less than the flow rate reference value.

Example 60: The medical aspiration system of example 59, wherein modifying the duty cycle comprises increasing, in response to determining that the flow rate is less than the flow rate reference value by a first difference amount, the duty cycle by a first duty cycle change amount, wherein modifying the duty cycle comprises increasing, in response to determining that the flow rate is less than the flow rate reference value by a second difference amount that is greater than the first difference amount, the duty cycle by a second duty cycle change amount that is greater than the first duty cycle change amount, wherein increasing the duty cycle by the first duty cycle change amount comprises decreasing an amount of time the valve is in the first operational state by a first amount of time and increasing an amount of time the valve is in the second operational state by the first amount of time, and wherein increasing the duty cycle by the second duty cycle change amount comprises decreasing an amount of time the valve is in the first operational state by a second amount of time that is greater than the first amount of time and increasing an amount of time the valve is in the second operational state by the second amount of time.

Example 61: The medical aspiration system of any one of examples 55 through 60, wherein determining the flow rate comprises determining the flow rate based on a flow rate measurement from a flow rate sensor, wherein the flow rate reference value is greater than a flow rate noise amount of the flow rate sensor.

Example 62: The medical aspiration system of any one of examples 55 through 61, wherein determining the flow rate comprises determining the flow rate based on a flow rate measurement from a flow rate sensor, and wherein the flow rate reference value is less than or equal to a flow rate noise amount of the flow rate sensor, wherein the flow rate is a first flow rate, wherein the control circuitry is further configured to: control the valve to be in the second operational state for a predetermined amount of time; determine, using the flow rate sensor and within the predetermined amount of time, a second flow rate of the fluid within the catheter lumen; and control the valve to be in the first operational state or to be in the second operational state to control the suction force applied to the catheter lumen based on the second flow rate and the flow rate reference value.

Example 63: The medical aspiration system of example 62, wherein the duty cycle is a first duty cycle, wherein the control circuitry is further configured to: determine a second duty cycle of the first and second operational states based on a difference between the second flow rate and the flow rate reference value; and control the valve to be in the first operational state or to be in the second operational state according to the second duty cycle.

Example 64: The medical aspiration system of any one of examples 55 through 63, wherein controlling the valve to be in the first operational state or the second operational state according to the duty cycle comprises controlling the valve according to at least one of proportional-integral-derivative (PID) control loop or an adaptive algorithm.

Example 65: The medical aspiration system of any one of examples 55 through 64, wherein the control circuitry is further configured to: determine an amount to partially open or close the valve based on the difference between the flow rate and the flow rate reference value; and control the valve to open and close according to the partial amount.

Example 66: The medical aspiration system of any one of examples 55 through 65, wherein the control circuitry is further configured to determine the flow rate reference value based on a size of the catheter lumen.

Example 67: The medical aspiration system of any one of examples 55 through 66, further including: a suction source; and an elongated body defining the catheter lumen.

Example 68: A medical device for aspirating material from a patient, the device including: a suction source; an aspiration catheter defining a lumen fluidically coupled to the suction source; a valve configured to open or close to control a suction force applied to the catheter lumen; and control circuitry configured to control the valve to open or close based on a flow rate and a flow rate reference value.

Various aspects of the disclosure have been described. These and other aspects are within the scope of the following claims.

What is claimed is:

1. A method comprising:
determining, by control circuitry, a flow rate of a fluid within a catheter lumen fluidically coupled to a valve;
comparing, by the control circuitry, the flow rate to a first flow rate threshold and a second flow rate threshold less than the first flow rate threshold;
in response to determining that the flow rate is greater than or equal to the first flow rate threshold, controlling, by the control circuitry, the valve to be in a first operational state; and
in response to determining that the flow rate is less than or equal to the second flow rate threshold, controlling, by the control circuitry, the valve to be in a second operational state.

2. The method of claim 1, wherein the first operational state comprises a substantially closed valve state in which the valve enables application of a relatively low suction force to the catheter lumen, and wherein the second operational state comprises a substantially open valve state in which the valve enables application of a relatively high suction force to the catheter lumen.

3. The method of claim 1, further comprising:
determining, by the control circuitry, that a first predetermined amount of time has passed with the valve in the first operational state; and
in response to determining, by the control circuitry, that the first predetermined amount of time has passed with the valve in the first operational state, controlling the valve to be in the second operational state for a second predetermined amount of time.

4. The method of claim 1, further comprising:
in response to determining that the flow rate is less than the first flow rate threshold:
determining, by the control circuitry and at a flow measurement sampling rate, a subsequent flow rate of the fluid within the catheter lumen;
determining, by the control circuitry and at the flow measurement sampling rate, whether the subsequent flow rate of the fluid within the catheter lumen is greater than the second flow rate threshold; and
in response to determining that the subsequent flow rate is greater than the second flow rate threshold, controlling, by the control circuitry, the valve to be in a first operational state.

5. The method of claim 1, wherein controlling the valve to be in the first operational state comprises controlling the valve to be in the first operational state for a first predetermined amount of time, and wherein controlling the valve to be in the second operational state comprises controlling the valve to be in the second operational state for a second predetermined amount of time, the method further comprising:
in response to determining that the flow rate is less than the first flow rate threshold and greater than the second flow rate threshold:
determining, by the control circuitry and at a flow measurement sampling rate, a subsequent flow rate of the fluid within the catheter lumen; and
controlling, by the control circuitry, the valve to alternatingly be in the first operational state for a third amount of time and to be in the second operational state for a fourth amount of time while the subsequent flow rate of the fluid within the catheter lumen is less than the first flow rate threshold and greater than the second flow rate threshold during the third or fourth amounts of time.

6. The method of claim 1, wherein controlling the valve to be in the first operational state comprises controlling the valve to be in the first operational state for a first predetermined amount of time, the method further comprising:
controlling, by the control circuitry, the valve to be in the second operational state for a second predetermined amount of time;
determining, by the control circuitry and at a flow measurement sampling rate, that the flow rate during the first or second predetermined amount of time is greater than or equal to the first flow rate threshold; and
controlling, by the control circuitry, the valve to be in the first operational state in response to determining the flow rate during the first or second predetermined amount of time is greater than or equal to the first flow rate threshold.

7. The method of claim 1, wherein controlling the valve to be in the first operational state comprises controlling the valve to be in the first operational state for a first predetermined amount of time, the method further comprising:
controlling, by the control circuitry, the valve to be in the second operational state for a second predetermined amount of time;
determining, by the control circuitry and at a flow measurement sampling rate, that the flow rate during the first or second predetermined amount of time is less than the first flow rate threshold; and
controlling, by the control circuitry, the valve to alternatingly be in the first operational state for a third amount of time and to be in the second operational state for a fourth amount of time while a subsequent flow rate of the fluid within the catheter lumen is less than the first flow rate threshold and greater than the second flow rate threshold during the third or fourth amounts of time.

8. The method of claim 1, wherein the flow rate includes a first flow rate, the method further comprising:
controlling, by the control circuitry, the valve to alternatingly be in the first and second operational states for a plurality of valve cycles, wherein each valve cycle of the plurality of valve cycles comprises:
the first operational state in which the valve is closed to reduce a suction force applied to the catheter lumen for a first predetermined amount of time; and
the second operational state in which the valve is open to increase the suction force applied to the catheter lumen for a second predetermined amount of time;
determining, by the control circuitry, a subsequent flow rate during or after of the second predetermined amount of time; and
determining, by the control circuitry, at least one of the first flow rate threshold or the second flow rate threshold based on the subsequent flow rate during the plurality of valve cycles.

9. A medical aspiration system comprising:
a valve configured to open or close to control a suction force applied to a catheter lumen; and
control circuitry configured to:
determine a flow rate of a fluid within the catheter lumen;
compare the flow rate to a first flow rate threshold and a second flow rate threshold less than the first flow rate threshold;
in response to determining that the flow rate is greater than or equal to the first flow rate threshold, control the valve to be in a first operational state; and
in response to determining that the flow rate is less than or equal to the second flow rate threshold, control the valve to be in a second operational state.

10. The medical aspiration system of claim 9, wherein the first operational state comprises a substantially closed valve state in which the valve enables application of a relatively low suction force to the catheter lumen, and wherein the second operational state comprises a substantially open valve state in which the valve enables application of a relatively high suction force to the catheter lumen.

11. The medical aspiration system of claim 9, wherein the control circuitry is further configured to:
determine that a first predetermined amount of time has passed with the valve in the first operational state; and
in response to determining that the first predetermined amount of time has passed with the valve in the first operational state, control the valve to be in the second operational state for a second predetermined amount of time.

12. The medical aspiration system of claim 11, wherein the first predetermined amount of time is about 950 milliseconds (ms), wherein the second predetermined amount of time is about 50 ms, and wherein determining the flow rate comprises measuring the flow rate after second predetermined amount of time.

13. The medical aspiration system of claim 9, wherein the control circuitry is further configured to:
in response to determining that the flow rate is less than the first flow rate threshold:
determine, at a flow measurement sampling rate, a subsequent flow rate of the fluid within the catheter lumen;
determine, at the flow measurement sampling rate, whether the subsequent flow rate of the fluid within the catheter lumen is greater than the second flow rate threshold; and
in response to determining that the subsequent flow rate is greater than the second flow rate threshold, control the valve to be in a first operational state.

14. The medical aspiration system of claim 9, wherein determining the flow rate comprises directly measuring the flow rate of the fluid within the catheter lumen.

15. The medical aspiration system of claim 9, wherein determining the flow rate comprises indirectly measuring the flow rate of the fluid within the catheter lumen by at least measuring the flow rate of the fluid within a volume fluidically coupled to the catheter lumen.

16. The medical aspiration system of claim 9, wherein controlling the valve to be in the first operational state comprises controlling the valve to be in the first operational state for a first predetermined amount of time, wherein controlling the valve to be in the second operational state comprises controlling the valve to be in the second operational state for a second predetermined amount of time, wherein the control circuitry is further configured to:
in response to determining that the flow rate is less than the first flow rate threshold and greater than the second flow rate threshold:
determine, at a flow measurement sampling rate, a subsequent flow rate of the fluid within the catheter lumen; and
control the valve to alternatingly be in the first operational state for a third amount of time and to be in the second operational state for a fourth amount of time while the subsequent flow rate of the fluid within the catheter lumen is less than or equal to the first flow rate threshold and greater than or equal to the second flow rate threshold during the third or fourth amounts of time.

17. The medical aspiration system of claim 16, wherein the third amount of time and the fourth amount of time are substantially equal.

18. The medical aspiration system of claim 16, wherein the flow measurement sampling rate comprises time intervals that are less than the first predetermined amount of time, the second predetermined amount of time, the third amount of time, and the fourth amount of time.

19. The medical aspiration system of claim 9, wherein controlling the valve to be in the first operational state comprises controlling the valve to be in the first operational state for a first predetermined amount of time, and wherein the control circuitry is further configured to:
control the valve to be in the second operational state for a second predetermined amount of time;
determine, at a flow measurement sampling rate, that the flow rate during the first or second predetermined amount of time is greater than or equal to the first flow rate threshold; and
control the valve to be in the first operational state in response to determining the flow rate during the first or second predetermined amount of time is greater than or equal to the first flow rate threshold.

20. The medical aspiration system of claim 9, wherein controlling the valve to be in the first operational state comprises controlling the valve to be in the first operational state for a first predetermined amount of time, and wherein the control circuitry is further configured to:
control the valve to be in the second operational state for a second predetermined amount of time;
determine, at a flow measurement sampling rate, that the flow rate during the first or second predetermined amount of time is less than the first flow rate threshold; and
determine, at the flow measurement sampling rate, a subsequent flow rate of the fluid within the catheter lumen; and
control the valve to alternatingly be in the first operational state for a third amount of time and to be in the second operational state for a fourth amount of time while the subsequent flow rate of the fluid within the catheter lumen is less than or equal to the first flow rate threshold and greater than or equal to the second flow rate threshold during the third or fourth amounts of time.

21. The medical aspiration system of claim 20, wherein the flow measurement sampling rate comprises time intervals that are less than the first predetermined amount of time, the second predetermined amount of time, the third amount of time, and the fourth amount of time.

22. The medical aspiration system of claim 20, wherein the third amount of time and the fourth amount of time are substantially equal.

23. The medical aspiration system of claim 9, wherein the flow rate includes a first flow rate, and wherein the control circuitry is further configured to:
control the valve to alternatingly be in the first and second operational states for a plurality of valve cycles, wherein each valve cycle of the plurality of valve cycles comprises:
the first operational state in which the valve is closed to reduce the suction force applied to the catheter lumen for a first predetermined amount of time; and
the second operational state in which the valve is open to increase the suction force applied to the catheter lumen for a second predetermined amount of time;
determine a subsequent flow rate during or after of the second predetermined amount of time; and
determine at least one of the first flow rate threshold or the second flow rate threshold based on a second flow rate during the plurality of valve cycles.

24. The medical aspiration system of claim 23, wherein the control circuitry is further configured to:
determine, during a valve cycle of the plurality valve cycles, that the subsequent flow rate is less than the first flow rate threshold; and
decrease the first flow rate threshold to be less than or equal to the subsequent flow rate in response to determining that the subsequent flow rate is less than the first flow rate threshold.

25. The medical aspiration system of claim 23, wherein the control circuitry is further configured to:
determine, during a valve cycle of the plurality valve cycles, that the subsequent flow rate is greater than a maximum flow rate; and
increasing the first flow rate threshold based on the subsequent flow rate.

26. The medical aspiration system of claim 23, wherein the control circuitry is further configured to determine a size of the catheter lumen based on a maximum flow rate of the plurality of cycles.

27. A medical device for aspirating material from a patient, the device comprising:
a suction source;
an aspiration catheter defining a lumen fluidically coupled to the suction source;
a valve configured to open or close to control a suction force applied to the catheter lumen; and
control circuitry configured to control the valve to open or close based on a flow rate, a first flow rate threshold, and a second flow rate threshold.
